(19) United States
(12) Patent Application Publication
(10) Pub. No.: US 2013/0225694 A1
(43) Pub. Date: Aug. 29, 2013

(54) COMPOSITIONS AND METHODS OF INHIBITING RETINAL DEGENERATION

(71) Applicants: Robert E. Anderson, Edmond, OK (US); Nawajes A. Mandal, Edmond, OK (US); Jian-Xing Ma, Edmond, OK (US); Gennadiy Moiseyev, Edmond, OK (US); Robert A. Floyd, Oklahoma, OK (US)

(72) Inventors: Robert E. Anderson, Edmond, OK (US); Nawajes A. Mandal, Edmond, OK (US); Jian-Xing Ma, Edmond, OK (US); Gennadiy Moiseyev, Edmond, OK (US); Robert A. Floyd, Oklahoma, OK (US)

(73) Assignees: Oklahoma Medical Research Foundation, Oklahoma City, OK (US); The Board of Regents of the University of Oklahoma, Oklahoma City, OK (US)

(21) Appl. No.: 13/775,771

(22) Filed: Feb. 25, 2013

Related U.S. Application Data
(60) Provisional application No. 61/602,981, filed on Feb. 24, 2012.
(60) Provisional application No. 61/755,786, filed on Jan. 23, 2013.

(51) Int. Cl. A61K 31/04

(52) U.S. Cl. CPC A61K 31/04
USPC 514/741; 435/375

(57) ABSTRACT

Nitrone-based compositions are disclosed that may be utilized for the prevention and treatment of a variety of ophthalmic diseases or conditions where RPE65 protein isomerohydrolase is implicated. Methods of production and use of said nitrone-based compositions, as well as pharmaceutical and ophthalmic compositions containing same, are disclosed.

### CROSS REFERENCE TO RELATED APPLICATIONS/INCORPORATION BY REFERENCE STATEMENT

This application claims benefit under 35 USC 119(e) of provisional applications U.S. Ser. Nos. 61/602,981, filed Feb. 24, 2012; and 61/755,786, filed Jan. 23, 2013. The entire contents of the above-referenced patent applications are hereby expressly incorporated herein by reference.

### STATEMENT REGARDING FEDERALLY FUNDED RESEARCH OR DEVELOPMENT

Not Applicable.

### BACKGROUND

The presently disclosed and claimed inventive concept(s) relates, in general, to compositions and methods of inhibiting retinal degeneration. More particularly, the presently disclosed and claimed inventive concept(s) is related to methods of inhibiting retinal degeneration utilizing nitrone-based compositions.

At present, approximately 1.75 million Americans have age-related macular degeneration (AMD). The atrophic, non-exudative, or drusenoid macular degeneration, collectively called ‘dry AMD’, accounts for about 90% of all AMD cases. Dry AMD does not usually cause complete loss of vision, but significantly impairs central vision required for reading, driving, and other visually detailed tasks. A significant proportion of advanced dry AMD transforms to ‘wet’ or neovascular AMD, which is vision threatening. Anti-angiogenic therapies have been developed for wet AMD, but there is no proven therapy for dry AMD. One of the hallmarks of AMD and several other retinal degenerative diseases is the presence of lipofuscin in the retinal pigmented epithelium (RPE). A major component of lipofuscin is A2E, a bisretinoid that is a product of the condensation of all-trans-retinal with the membrane lipid phosphatidylethanolamine. All-trans-retinal is generated when the visual pigment rhodopsin absorbs a photon of light. It is a normal byproduct of visual excitation, and is transported to the RPE, where it is converted to 11-cis-retinal, which is returned to the retina, where it is incorporated into rhodopsin. The photobleaching of rhodopsin, movement of all-trans-retinal to the RPE, its enzymatic conversion to 11-cis-retinal, the movement of 11-cis-retinal back to the retina, and its incorporation into rhodopsin is known as the “Visual Cycle”. With age, and in some inherited diseases, there is an accumulation of lipofuscin (and A2E) in the RPE. A2E has been clearly demonstrated to be toxic to RPE and other cells, and its accumulation in the RPE is thought to be one of the events that ultimately leads to the death of RPE cells. One strategy currently being studied to slow the accumulation of A2E in the RPE is to slow down the Visual Cycle, which will reduce the amount of 11-cis-retinal available for formation of rhodopsin, and thus reduce the amount of all-trans-retinal that is produced by light. This approach has been successful in animal models where lipofuscin and A2E accumulate. Several drugs are currently being tested in humans, but these drugs have significant side effects.

As described above, the retinoid visual cycle is a multistep process for the recycling of 11-cis-retinal (the chromophore of both rod and cone visual pigments) and is essential for regeneration of visual pigment and maintenance of normal vision. The key step of the visual cycle is the hydrolysis-isomerization of all-trans-retinyl ester to 11-cis-retinol. Previously, the inventors have established that the enzyme that catalyzes the isomerization step is RPE65 (retinal pigment epithelium-specific protein of 65 kDa) isomerohydrolase, which is expressed in the RPE. Intact RPE65 function is essential for vision, because mutations in the RPE65 gene have been reported to cause several forms of inherited retinal dystrophies. Moreover, no 11-cis-retinoids were detected in the RPE65^{-/-} mice, suggesting that RPE65 is the only enzyme that can produce 11-cis-retinoids in the eye. RPE65^{-/-} mice were found to be completely protected against light-induced apoptosis. The inhibition of RPE65 isomerohydrolase activity can be beneficial for maintenance of normal vision.

Thus there is a need for new and improved compositions and methods for inhibition of RPE65 isomerohydrolase activity in RPE cells. The compositions and methods described herein are directed toward this end.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates the effect of PBN on protection against light-induced retinal degeneration. (A) Retinal rod photoreceptor function was measured by ERG 7 days after light-exposure. Full-field dark-adapted ERGs were recorded for flashes of increasing intensities (−2.3-2.7 log cd·s/m²). Scotopic a-wave and b-wave responses (mean±SE, n=6-10 rats) is presented NLD, no light-damage; and LD, light-damaged. (B) After ERG recordings, eyes were harvested for histology, marked for orientation and fixed. Five μm sections were cut along the vertical meridian through the optic nerve and stained with H&E. Representative sections from each treatment were imaged from the superior-central retina, which is specifically affected by exposure to damaging light. Panels on the right represent higher magnifications of the superior-central-retina. I-II, NLD; III-IV, Saline-before-LD; V-VI, PBN-before-LD; VII-VIII, PBN-after-LD; IX-X, S-PBN-before-LD. (C) Quantitative morphometric measurement of outer nuclear layer (ONL) thickness was measured from H&E stained slides along the vertical meridian from superior to inferior retina (n=8-10). ONH, Optic nerve head.

FIG. 2 illustrates the effect of the time of PBN administration on the ability to protect against light-induced retinal degeneration. (A) Rod photoreceptor function was measured by ERG 7 days after light-exposure. (B) Representative H&E sections as described in FIG. 1B. I-II, PBN 3 h before LD; III-IV, PBN 6 h before LD; V-VI, PBN 12 h before LD; VII-VIII, PBN 16 h before LD; IX-X, PBN 24 h before LD. (C) Quantitative morphometry of ONL thickness of the samples described in A and B (n=6-8).

FIG. 3 illustrates that PBN suppresses recovery of rod responses after bleaching. Recovery of rod photoresponses after bleaching (2.3 log cd/m² for 2 minutes) in PBN- (grey square, n=4) and saline-treated rats (solid circle, n=4). A-wave responses before and after bleaching were elicited by 2.3 log cd·s/m² flashes and were normalized to the amplitude of the initial, dark-adapted response. PBN administration significantly reduced the recovery of rod responses at the indicated time points (*P≦0.05, **P≦0.005; unpaired t-test, n=4).

FIG. 4 illustrates that PBN administration does not inhibit cone function. PBN or saline were administered 60 minutes prior to recording single-flash photopic and flicker-flash ERGs under a steady adapting field of 1.7 log cd/m². Representative single-flash photopic (A) and flicker flash (B) responses are indicated. (C) Quantitative analysis of single-flash and flicker-flash ERG responses (n=4), indicated that PBN did not suppress cone function under conditions when rod recovery is inhibited. No significant differences were found between PBN and saline for any cone responses (unpaired t-test, n=4).

FIG. 5 illustrates that PBN administration inhibits rhodopsin regeneration. Rats were dark-adapted overnight and injected with PBN or PBS (vehicle) immediately prior to a complete bleach for 2 hours in room light (˜400 lux). Rats were returned to darkness for the indicated time points and retinas were collected for spectrophotometric determination of rhodopsin content. Values represent the mean nmol rhodopsin/eye, and the error bars represent the SEM. PBN significantly inhibited rhodopsin regeneration at the indicated time points (**P≦0.005; unpaired t-test, n≧4). No significant differences were observed between fully dark-adapted rats injected with either PBN (1.38±0.14 nmol/eye) or PBS (1.41±0.07 nmol/eye). The dotted line represents the mean rhodopsin content of fully dark-adapted rats injected with or without PBN.

FIG. 6 illustrates that PBN does not inhibit retinol dehydrogenase activity. Retinal RDH activity was measured in the presence and absence of PBN with increasing concentrations of substrate. The representative experiment shown in (A) was repeated 2 times with similar results. Histograms of the relative RDH activities at three different substrate concentrations and 2 mM PBN are shown in (B). In this graph, the RDH activity without PBN represents 100%. No statistically significant differences were found between RDH activities in absence and in presence of PBN.

FIG. 7 illustrates that PBN inhibits retinol isomerohydrolase activity in vitro. RPE microsomal protein (50 μg) was incubated with 0.2 μM all-trans-[³H]-retinol with and without PBN for 1 hour at 37° C. The generated retinoids were analyzed by normal phase HPLC. HPLC elution profiles (A) without PBN; (B) with 1 mM PBN; and (C) with 5 mM S-PBN. Peak 1, retinyl esters; peak 2, 11-cis-retinol; and peak 3, all-trans-retinol. (D) PBN concentration-dependent inhibition of 11-cis-retinol generation (mean±SEM, n=3).

FIG. 8 illustrates uncompetitive inhibition of RPE65 isomerohydrolase by PBN in the liposome-based isomerohydrolase assay. Lineweaver-Burk plots of 11-cis-retinol generated by RPE65. Liposomes with increasing concentrations of all-trans-retinyl palmitate (S) were incubated with equal amounts (125 μg) of chicken RPE65 expressed in 293A cells, with (▪) and without (∘) PBN (0.1 mM).

FIG. 9 illustrates the calculation of I_{C50}) values for two difluoro-PBN analogs.

FIG. 10 graphically illustrates the effects of three PBN analogs of rhodopsin regeneration. Rhodopsin content was measured as nmole/retina following the procedure as in Mandal et al. (J Biol. Chem. 2011. 286 (37):32491-501). After injection with PBN derivatives or vehicles (saline), rats were left in room light for 2 hours (light adapted or LA) and then moved to dark. At 2.5 hours dark adaptation (DA), retinas were harvested. DA-Control, Dark adapted (12 h) control retina without any treatment to show normal rhodopsin content. LA-Control, Light adapted (for 2 hours) control retina shows complete bleach or no measurable rhodopsin.

FIG. 11 graphically illustrates electroretinographic (ERG) A-wave analysis of retinal damage/protection observed for the three PBN analogs. ERG responses were recorded for two flash stimuli at intensities of 4 and 400 cd·sec/m². A-wave represents the responses obtained directly from photoreceptor cells. The dim flash (4 cd·sec/m²) stimulated the rod photoreceptor cells, and the bright flash (400 cd·sec/m²) stimulated both rod and cone photoreceptors. Therefore the blue bars represent only rod responses, and the red bars represent mixed response from both rod and cone photoreceptors. NLD represents Non-light-damaged group, and LD represents light-damaged groups.

FIG. 12 graphically illustrates ERG B-wave analysis of retinal damage/protection observed for the three PBN analogs. ERG responses were recorded for two flash stimuli at intensities of 4 and 400 cd·sec/m². The B-wave represents the amplification of the A-wave response obtained from secondary neurons in the retina. The dim flash (4 cd·sec/m²) stimulated the rod photoreceptor cells, and the bright flash (400 cd·sec/m²) stimulated both rod and cone photoreceptors. Therefore, the blue bars represent only rod responses, and the red bars represent mixed response from both rod and cone photoreceptors. NLD represents Non-light-damaged group and LD represents light-damaged groups.

FIG. 13 contains a photo illustrating a histological analysis of rat eyes treated with the PBN analogs. Eyes were harvested, and the orientation marked; the eyes were then fixed, embedded in paraffin, cut through vertical meridian, and stained with H&E. Outer nuclear layer (ONL) thickness was measured in the central retina inferior to superior through optic nerve head (ONH). ONL thickness represents viable photoreceptor cells. The value is ‘0’ at ONH, as there are no photoreceptor cells at the ONH.

FIG. 14 graphically illustrates that 4-F-PBN provided significant protection of retinal photoreceptors. Vehicle LD, vehicle or saline treated rats; NLD is non-light-damage control. PBN LD, PBN treatment.

FIG. 15 graphically illustrates that 4-CF₃-PBN provided significant protection of retinal photoreceptors. Vehicle LD, vehicle or saline treated rats; NLD is non-light-damage control. PBN LD, PBN treatment.

FIG. 16 graphically illustrates that 4-Me-PBN provided significant protection of retinal photoreceptors. Vehicle LD, vehicle or saline treated rats; NLD is non-light-damage control. PBN LD, PBN treatment.

FIG. 17 graphically illustrates that PBN administration 0.5-12 hours before exposure to damaging light protects cone function, as measured by single-flash photopic ERG. Cone photoreceptor function was measured by single-flash photopic ERG 7 days after light-exposure. A single strobe flash stimulus of 3.3 log cd·s/m² was presented to dilated, light-adapted (5 minutes at 1.7 log cd/m²) rats. The amplitude of the cone b-wave was measured from the trough of the a-wave to the peak of the b-wave. NLD, No-light-damaged; LD, light-damaged; S, saline; P, PBN. (* significant at p<0.01).

FIG. 18 shows rhodopsin regeneration after topical application of PBN eye drops. Panel A: Application scheme and harvesting of the eye. 3 h group received 2 applications and 6 h group received 3 applications. Panel B: Rhodopsin content was measured as pmole/eye. In 1 h in the dark, untreated eyes (NT-LA 1 h DA, no treatment-light adapted followed by 1 hour dark adaptation) and vehicle-treated eyes recovered 75-80% rhodopsin [dark-adapted (DA) control is 100%], whereas 3 h PBN recovered only 43% and 6 h PBN only 29%. (***P<0.0001 compared to vehicle, n=8; ##p<0.005 between 3 h and 6 h group, n=8).

FIG. 19 shows a measurement of PBN in the posterior eye cup by MS-MS analysis using nanospray direct infusion (TriVersa NanoMate®, Advion, Inc., Ithaca, N.Y.) with a TSQ Ultra triple quadrupole mass spectrometer (Thermo Fisher Scientific, Inc., San Jose, Calif.).

FIG. 20 shows that topically-applied PBN protects mouse retina from light-damage (LD). After topical application of PBN, light-damage was done at 3,000 lux for 6 h. ERG response were recorded five days after light-damage. Scotopic A-wave amplitudes measured at increasing flash intensity and presented in the figure to show rod function. Two vehicles were used to solubilize and make 10% PBN eye drop formulation for topical application on mouse eye, vehicle 1 (V-1) and vehicle 2 (V-2). Upon light-damage, A-wave amplitudes decreased significantly in untreated control eyes (No PBN_LD), however, significant restoration of rod function was observed in PBN treated eyes, in which PBN was solubilized either in V1 or V2 (P-1_LD and P-2_LD). Vehicles showed slight protection (V-1_LD and V-2_LD) but PBN protection is higher than vehicles (* p<0.05, n=6). Vehicle 2 did not induce as much of a protective response by itself as that seen when Vehicle 1 was used.

### DETAILED DESCRIPTION

Before explaining at least one embodiment of the inventive concept(s) in detail by way of exemplary drawings, experimentation, results, and laboratory procedures, it is to be understood that the inventive concept(s) is not limited in its application to the details of construction and the arrangement of the components set forth in the following description or illustrated in the drawings, experimentation and/or results. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed and claimed inventive concept(s) shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2nd ed., Cold Spring Harbor Laboratory Press, Cold Spring Harbor, N.Y. (1989) and Coligan et al. Current Protocols in Immunology (Current Protocols, Wiley Interscience (1994)), which are incorporated herein by reference. The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

All patents, published patent applications, and non-patent publications mentioned in the specification are indicative of the level of skill of those of ordinary skill in the art to which the presently disclosed and claimed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions and/or methods disclosed and claimed herein can be made and executed without undue experimentation in light of the present disclosure. While the compositions and methods of the presently disclosed and claimed inventive concept(s) have been described in terms of preferred embodiments, it will be apparent to those of ordinary skill in the art that variations may be applied to the compositions and/or methods and in the steps or in the sequence of steps of the method described herein without departing from the concept, spirit and scope of the presently disclosed and claimed inventive concept(s). All such similar substitutes and modifications apparent to those of ordinary skill in the art are deemed to be within the spirit, scope and concept of the inventive concept(s) as defined by the appended claims.

As utilized in accordance with the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects. The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

The term “isolated” as used herein means that a biological material, such as but not limited to a nucleic acid or protein, has been removed from its original environment in which it is naturally present. For example, a polynucleotide present in a plant, mammal or animal is present in its natural state and is not considered to be isolated. The same polynucleotide separated from the adjacent nucleic acid sequences in which it is naturally inserted in the genome of the plant or animal is considered as being “isolated.”

The term “isolated” is not meant to exclude artificial or synthetic mixtures with other compounds, or the presence of impurities which do not interfere with the biological activity and which may be present, for example, due to incomplete purification, addition of stabilizers or mixtures with pharmaceutically acceptable excipients, and the like.

“Isolated polypeptide” or “isolated protein” as used herein means a polypeptide or protein which is substantially free of those compounds that are normally associated with the polypeptide or protein in a natural state, including but not limited to, other proteins or polypeptides, nucleic acids, carbohydrates, lipids and the like.

The term “purified” as used herein means at least one order of magnitude of purification is achieved compared to the starting material or of the natural material, for example but not by way of limitation, two, three, four or five orders of magnitude of purification of the starting material or of the natural material. Thus, the term “purified” as utilized herein does not necessarily mean that the material is 100% purified, and therefore such term does not exclude the presence of other material(s) present in the purified composition.

Throughout the specification and claims, unless the context requires otherwise, the terms “substantially” and “about” will be understood to not be limited to the specific terms qualified by these adjectives/adverbs, but allow for minor variations and/or deviations that do not result in a significant impact thereto. For example, in certain instances the term “about” is used to indicate that a value includes the inherent variation of error for the device, the method being employed to determine the value and/or the variation that exists among study subjects. Similarly, the term “substantially” may also relate to 80% or higher, such as 85% or higher, or 90% or higher, or 95% or higher, or 99% or higher, and the like.

The terms “analog” and “derivative” are used herein interchangeably and refer to a substance which comprises the same basic carbon skeleton and carbon functionality in its structure as a given compound, but can also contain one or more substitutions thereto.

The term “substitution” as used herein will be understood to refer to the replacement of at least one substituent on a compound with a residue R. In certain non-limiting embodiments, R may include H, hydroxyl, thiol, a halogenid selected from fluoride, chloride bromide or iodite, a C1-C8 compound selected one of the following: linear, branched or cyclic alkyl, optionally substituted, and linear branched or cyclic alkenyl, wherein the optional substitutents are selected from one or more alkenylalkyl, alkynylalkyl, cycloalkyl, cycloalkenylalkyl, arylalkyl, heteroarylalkyl, heterocyclealkyl, optionally substituted heterocycloalkenylalkyl, arylcycloalkyl, and arylheterocycloalkyl, each of which is optionally substituted wherein the optional substitutents are selected from one or more of alkenylalkyl, alkynylalkyl, cycloalkyl, cyclalkenylalkyl, arylalkyl, alkylaryl, heteroarylalkyl, heterocyclealkyl, optionally substituted heterocycloalkenylalkyl, arylcycloalkyl, and arylheterocyclalkyl, phenyl, cyano, hydroxyl, alkyl, aryl, cycloalkyl, cyano, alkoxy, alkylthio, amino, —NH (alkyl), —NH(cycloalkyl)₂, carboxy and —C(O))-alkyl.

The term “patient” includes human and veterinary subjects. In certain embodiments, a patient is a mammal. In certain other embodiments, the patient is a human.

“Treatment” refers to both therapeutic treatment and prophylactic or preventative measures. Those in need of treatment include, but are not limited to, individuals already having a particular condition or disorder as well as individuals who are at risk of acquiring a particular condition or disorder (e.g., those needing prophylactic/preventative measures). The term “treating” refers to administering an agent to a patient for therapeutic and/or prophylactic/preventative purposes.

A “therapeutic agent” refers to an agent that may be administered in vivo to bring about a therapeutic and/or prophylactic/preventative effect.

An “ocular disorder” is any ocular condition that would benefit from treatment with the compositions disclosed herein. This includes chronic and acute ocular disorders or diseases including those pathological conditions which predispose the mammal to the ocular disorder in question.

“Mammal” for purposes of treatment refers to any animal classified as a mammal, including human, domestic and farm animals, nonhuman primates, and zoo, sports, or pet animals, such as dogs, horses, cats, cows, etc.

The term “effective amount” refers to an amount of a biologically active molecule or conjugate or derivative thereof sufficient to exhibit a detectable therapeutic effect without undue adverse side effects (such as toxicity, irritation and allergic response) commensurate with a reasonable benefit/risk ratio when used in the manner of the presently disclosed and claimed inventive concept(s). The therapeutic effect may include, for example but not by way of limitation, inhibiting retinal degeneration by inhibiting toxic retinoid by-product accumulation, decreasing expression and/or activity of at least one retinal cell enzyme and/or slowing the visual cycle of the retina cell. The effective amount for a patient will depend upon the type of patient, the patient's size and health, the nature and severity of the condition to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

As used herein, the term “concurrent therapy” is used interchangeably with the terms “combination therapy” and “adjunct therapy”, and will be understood to mean that the patient in need of treatment is treated or given another drug for the disease/disorder in conjunction with the compositions of the presently disclosed and claimed inventive concept(s). This concurrent therapy can be sequential therapy, where the patient is treated first with one drug and then the other, or the two drugs are given simultaneously

The term “pharmaceutically acceptable” refers to compounds and compositions which are suitable for administration to humans and/or animals without undue adverse side effects such as toxicity, irritation and/or allergic response commensurate with a reasonable benefit/risk ratio.

The term “biologically active” refers to the ability of an agent to modify the physiological system of an organism. A molecule can be biologically active through its own functionalities, or may be biologically active based on its ability to activate or inhibit molecules having their own biological activity.

Generally, the compositions provided herein are administered in a therapeutically effective amount. The amount of the composition actually administered will typically be determined by a physician, in light of the relevant circumstances, including the condition to be treated, the chosen route of administration, the actual composition administered, the age, weight, and response of the individual patient, the severity of the patient's symptoms, and the like. The compositions of the presently disclosed and claimed inventive concept(s) may be administered to a patient by any method known in the art, including but not limited to, oral, topical, retrobulbar, subconjunctival, transdermal, parenteral, subcutaneous, intranasal, intramuscular, intraperitoneal, intravitreal, and intravenous routes, including both local and systemic applications. In addition, the compositions of the presently disclosed and claimed inventive concept(s) may be designed to provide delayed, controlled, extended, or sustained release using formulation techniques which are well known in the art. Depending on the intended route of delivery, the compounds provided herein are preferably formulated as either injectable or oral compositions or as salves, as lotions or as patches all for transdermal administration.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art. As before, the active compound in such compositions is typically a minor component, often being from about 0.05 to 10% by weight with the remainder being the injectable carrier and the like.

Transdermal compositions are typically formulated as a topical ointment or cream containing the active ingredient(s), generally in an amount ranging from about 0.01 to about 20% by weight, preferably from about 0.1 to about 20% by weight, preferably from about 0.1 to about 10% by weight, and more preferably from about 0.5 to about 15% by weight. When formulated as an ointment, the active ingredients will typically be combined with either a paraffinic or a water-miscible ointment base. Alternatively, the active ingredients may be formulated in a cream with, for example, an oil-in-water cream base. Such transdermal formulations are well-known in the art and generally include additional ingredients to enhance the dermal penetration of stability of the active ingredients or the formulation. All such known transdermal formulations and ingredients are included within the scope provided herein.

The compounds provided herein can also be administered by a transdermal device. Accordingly, transdermal administration can be accomplished using a patch either of the reservoir or porous membrane type, or of a solid matrix variety.

The term “ophthalmic composition” as used herein will be understood to refer to any composition for direct and local administration to an eye of a patient. Said composition may be administered topically to the eye or may be injected into the eye (i.e., intravitreal or intraocular injection). The ophthalmic composition may be provided in any form that allows local administration thereof to the eye, including but not limited to, solution, drops, mist/spray, plasters and pressure sensitive adhesives, ointment, lotion, cream, gel, lyophilized/spray-dried forms, and the like. The ophthalmic compositions of the invention typically vary according to the particular active agent (i.e., nitrone composition) used, the preferred drug release profile, the condition being treated, and the medical history of the patient. In addition, the ophthalmic compositions of the presently disclosed and claimed inventive concept(s) may be designed to provide delayed, controlled, extended, or sustained release using formulation techniques which are well known in the art.

The above-described components for orally, injectable, or topically administrable compositions are merely representative. Other materials as well as processing techniques and the like are set forth in Part 8 of Remington's The Science and Practice of Pharmacy, 21st edition, 2005, Publisher: Lippincott Williams & Wilkins, which is incorporated herein by reference.

The compounds of the presently disclosed and claimed inventive concept(s) can also be administered in sustained release forms or from sustained release drug delivery systems. A description of representative sustained release materials can be found in Remington's Pharmaceutical Sciences.

Particular abbreviations that may be utilized herein include: PBN, α-phenyl-N-tert-butyl nitrone; RPE65, retinal pigment epithelium-specific protein 65 kDa: SD, Sprague-Dawley rats; LRAT, lecithin retinol acyltransferase; RDH, retinol dehydrogenases; AMD, age-related macular degeneration; Cox-2, cyclooxigenase 2; iNos, nitric oxide synthase, inducible; AP-1, activator protein 1; NF-κB, nuclear factor kappa-B; ARVO, The Association for Research in Vision and Ophthalmology; PBS, phosphate-buffered saline; S-PBN, N-tert-butyl-α-(2-sulfophenyl)nitrone sodium salt; ERG, Electroretinography; ONL, outer nuclear layer; PR, photoreceptor; OPL, outer plexiform layer; INL, inner nuclear layer; IPL, inner plexiform layer; GCL, ganglion cell layer; RPE, retinal pigment epithelium cells; A2E, pyridinium bis-retinoid.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those of ordinary skill in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N.Y., 1962); and Wilen, Tables of Resolving Agents and Optical Resolutions p. 268 (E. L. Eliel, Ed., Univ. of Notre Dame Press, Notre Dame, Ind. 1972). The presently disclosed and claimed inventive concept(s) additionally encompasses compounds described herein as individual isomers substantially free of other isomers, and alternatively, as mixtures of various isomers.

When describing the presently disclosed and claimed inventive concept(s), which may include compounds, pharmaceutical compositions containing such compounds, and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” refers to a radical of a straight-chain or branched saturated hydrocarbon group having from 1 to 20 carbon atoms (“_{C1-20} alkyl”). In some embodiments, an alkyl group has 1 to 12 carbon atoms (“_{C1-12} alkyl”). In some embodiments, an alkyl group has 1 to 10 carbon atoms (“_{C1-10} alkyl”). In some embodiments, an alkyl group has 1 to 9 carbon atoms (“_{C1-9} alkyl”). In some embodiments, an alkyl group has 1 to 8 carbon atoms (“_{C1-8} alkyl”). In some embodiments, an alkyl group has 1 to 7 carbon atoms (“_{C1-7} alkyl”). In some embodiments, an alkyl group has 1 to 6 carbon atoms (“_{C1-6} alkyl”, also referred to herein as “lower alkyl”). In some embodiments, an alkyl group has 1 to 5 carbon atoms (“_{C1-5} alkyl”). In some embodiments, an alkyl group has 1 to 4 carbon atoms (“_{C1-4} alkyl”). In some embodiments, an alkyl group has 1 to 3 carbon atoms (“_{C1-3} alkyl”). In some embodiments, an alkyl group has 1 to 2 carbon atoms (“_{C1-2} alkyl”). In some embodiments, an alkyl group has 1 carbon atom (“_{C1} alkyl”). In some embodiments, an alkyl group has 2 to 6 carbon atoms (“_{C2-6} alkyl”). Examples of _{C1-6} alkyl groups include methyl (_{C1}), ethyl (_{C2}), n-propyl (_{C3}), isopropyl (_{C3}), n-butyl (_{C4}), tert-butyl (_{C4}), sec-butyl (_{C4}), iso-butyl (_{C4}), n-pentyl (_{C5}), 3-pentanyl (_{C5}), amyl (_{C5}), neopentyl (_{C5}), 3-methyl-2-butanyl (_{C5}), tertiary amyl (_{C5}), and n-hexyl (ₐ). Additional examples of alkyl groups include n-heptyl (_{C7}), n-octyl (_{C8}) and the like. Unless otherwise specified, each instance of an alkyl group is independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted _{C1-10} alkyl (e.g., —C_{H3}). In certain embodiments, the alkyl group is substituted _{C1-10} alkyl.

“Alkylene” refers to a substituted or unsubstituted alkyl group, as defined above, wherein two hydrogens are removed to provide a divalent radical. Exemplary divalent alkylene groups include, but are not limited to, methylene (—CH₂—), ethylene (—CH₂CH₂—), the propylene isomers (e.g., —CH₂CH₂CH₂— and —CH(CH₃)CH₂—) and the like.

“Alkenyl” refers to a radical of a straight-chain or branched hydrocarbon group having from 2 to 20 carbon atoms, one or more carbon-carbon double bonds, and no triple bonds (“C₂₋₂₀ alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂₋₁₀ alkenyl.

“Alkenylene” refers a substituted or unsubstituted alkenyl group, as defined above, wherein two hydrogens are removed to provide a divalent radical. Exemplary divalent alkenylene groups include, but are not limited to, ethenylene (—CH═CH—), propenylenes (e.g., —CH═CHCH₂— and —C(CH₃)═CH— and —CH═C(CH₃)—) and the like.

“Alkynyl” refers to a radical of a straight-chain or branched hydrocarbon group having from 2 to 20 carbon atoms, one or more carbon-carbon triple bonds, and optionally one or more double bonds (“C₂₋₂₀ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, i.e., unsubstituted (an “unsubstituted alkynyl”) or substituted (a “substituted alkynyl”) with one or more substituents; e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₁₀ alkynyl.

“Alkynylene” refers a substituted or unsubstituted alkynyl group, as defined above, wherein two hydrogens are removed to provide a divalent radical. Exemplary divalent alkynylene groups include, but are not limited to, ethynylene, propynylene, and the like.

“Aryl” refers to a radical of a monocyclic or polycyclic (e.g., bicyclic or tricyclic) 4n+2 aromatic ring system (e.g., having 6, 10, or 14 [symbol] electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₋₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particularly aryl groups include phenyl, naphthyl, indenyl, and tetrahydronaphthyl. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

In certain embodiments, an aryl group substituted with one or more of groups selected from halo, C₁-C₈ alkyl, C₁-C₈ haloalkyl, cyano, hydroxy, C₁-C₈ alkoxy, and amino.

Examples of representative substituted aryls include the following:

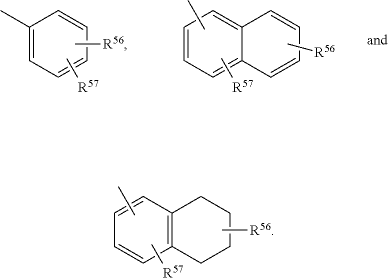

In these formulae, one of R⁵⁶ and R⁵⁷ may be hydrogen, and at least one of R⁵⁶ and R⁵⁷ is each independently selected from C₁-C₈ alkyl, C₁-C₈ haloalkyl, 4-10 membered heterocyclyl, alkanoyl, C₁-C₈ alkoxy, heteroaryloxy, alkylamino, arylamino, heteroarylamino, NR⁵⁸COR⁵⁹, NR⁵⁸SOR⁵⁹NR⁵⁸SO₂R⁵⁹, COOalkyl, COOaryl, CONR⁵⁸R⁵⁹, CONR⁵⁸OR⁵⁹, NR⁵⁸R⁵⁹, SO₂NR⁵⁸R⁵⁹, S-alkyl, SOalkyl, SO₂alkyl, Saryl, SOaryl, SO₂aryl; or R⁵⁶ and R⁵⁷ may be joined to form a cyclic ring (saturated or unsaturated) from 5 to 8 atoms, optionally containing one or more heteroatoms selected from the group N, O, or S. R⁶⁰ and R⁶¹ are independently hydrogen, C₁-C₈ alkyl, C₁-C₄ haloalkyl, C₃-C₁₀ cycloalkyl, 4-10 membered heterocyclyl, C₅-C₁₀ aryl, substituted C₆-C₁₀ aryl, 5-10 membered heteroaryl, or substituted 5-10 membered heteroaryl.

“Fused aryl” refers to an aryl having two of its ring carbons in common with a second aryl ring or with an aliphatic ring.

“Aralkyl” is a subset of alkyl and aryl, as defined herein, and refers to an optionally substituted alkyl group substituted by an optionally substituted aryl group.

“Heteroaryl” refers to a radical of a 5-10 membered monocyclic or bicyclic 4n+2 aromatic ring system (e.g., having 6 or 10 n electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

Non-limiting examples of representative heteroaryls include the following:

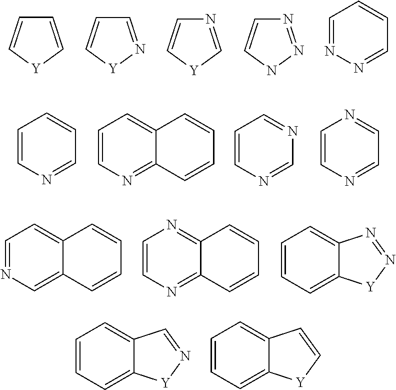

wherein each Y is selected from carbonyl, N, NR⁶⁵, O, and S; and R⁶⁵ is independently hydrogen, C₁-C₈ alkyl, C₃-C₁₀ cycloalkyl, 4-10 membered heterocyclyl, C₆-C₁₀ aryl, and 5-10 membered heteroaryl.

“Carbocyclyl” or “carbocyclic” refers to a radical of a non-aromatic cyclic hydrocarbon group having from 3 to 10 ring carbon atoms (“C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₈ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is a substituted C₃₋₁₀ carbocyclyl.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

“Heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or Spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

Particular examples of heterocyclyl groups are shown in the following illustrative examples:

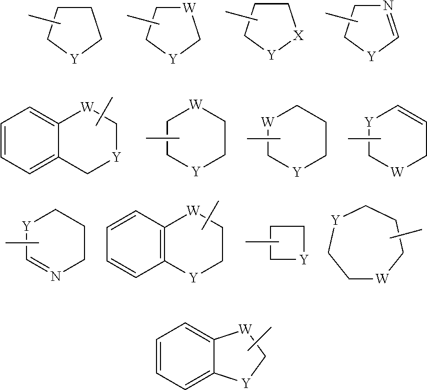

wherein each W is selected from CR⁶⁷, C(R⁶⁷)₂, NR⁶⁷, O, and S; and each Y is selected from NR⁶⁷, O, and O; and R⁶⁷ is independently hydrogen, C₁-C₈ alkyl, C₃-C₁₀ cycloalkyl, 4-10 membered heterocyclyl, C₆-C₁₀ aryl, 5-10 membered heteroaryl. These heterocyclyl rings may be optionally substituted with one or more substituents selected from the group consisting of the group consisting of acyl, acylamino, acyloxy, alkoxy, alkoxycarbonyl, alkoxycarbonylamino, amino, substituted amino, aminocarbonyl (carbamoyl or amido), aminocarbonylamino, aminosulfonyl, sulfonylamino, aryl, aryloxy, azido, carboxyl, cyano, cycloalkyl, halogen, hydroxy, keto, nitro, thiol, —S-alkyl, —S-aryl, —S(O)-alkyl, —S(O)-aryl, —S(O)₂-alkyl, and —S(O)₂-aryl. Substituting groups include carbonyl or thiocarbonyl which provide, for example, lactam and urea derivatives.

Turning now to particular embodiments of the presently claimed and disclosed inventive concept(s), compositions and methods of protecting at least one retinal cell from degeneration are disclosed. In the method, an effective amount of a nitrone composition is administered to at least one retinal cell to inhibit degeneration of the at least one retinal cell. In certain embodiments, the nitrone composition may be any of the inhibitors of RPE65 protein isomerohydrolase activity described in detail herein below or otherwise known in the art.

The presently disclosed and claimed inventive concept(s) also includes a method of treating a subject (such as but not limited to, a mammal) susceptible to or afflicted with a condition associated with RPE65 or RPE65 protein isomerohydrolase activity.

The presently disclosed and claimed inventive concept(s) also includes a method of decreasing the occurrence and/or severity of retinal degeneration associated with a retinal disorder/disease in a subject. In the method, an effective amount of the inhibitor of RPE65 protein isomerohydrolase activity is administered to the subject to inhibit degeneration of at least one retinal cell of the subject. The subject may be suffering from or predisposed to a retinal disorder/disease; non-limiting examples of retinal disorders/diseases (also referred to herein as ocular or ophthalmic diseases) include glaucoma, macular degeneration (including dry and wet forms of macular degeneration), age-related macular degeneration (AMD), Stargardt's disease, diabetic retinopathy, retinal detachment, retinal blood vessel (artery or vein) occlusion, retinitis pigmentosa, hemorrhagic retinopathy, retinopathy of prematurity, inflammatory retinal diseases, optic neuropathy, proliferative vitreoretinopathy, retinal dystrophy, ischemia-reperfusion related retinal injury, hereditary optic neuropathy, metabolic optic neuropathy, Sorsby's fundus dystrophy, Best disease, a retinal injury, a retinal disorder associated with viral and/or bacterial infection, a retinal disorder related to light overexposure, and retinal disorders associated with other degenerative diseases such as Alzheimer's disease, multiple sclerosis, and Parkinson's disease, or associated with AIDS or other diseases of the brain.

The presently disclosed and claimed inventive concept(s) is further directed to a method of treating an ophthalmic disease or disorder in a subject by inhibiting RPE65 protein isomerohydrolase activity in at least one ocular/retinal cell (such as, but not limited to, a retinal pigment epithelial cell) of the subject. In the method, an effective amount of the inhibitor of RPE65 protein isomerohydrolase activity is administered to the subject to inhibit degeneration of at least one retinal cell of the subject.

The presently disclosed and claimed inventive concept(s) also includes a method of treating a subject (such as but not limited to, a mammal) susceptible to or afflicted with a condition associated with RPE65 or RPE65 protein isomerohydrolase activity. In the method, an effective amount of the inhibitor of RPE65 protein isomerohydrolase activity is administered to the subject.

In any of the methods described herein above, the administration of the composition may result in a decrease in toxic retinoid by-product accumulation within the at least one retinal cell. The term “retinal cell” as used herein includes, but is not limited to, retinal pigment epithelial (RPE) cells and neural retinal cells. Thus, an additional embodiment of the presently disclosed and claimed inventive concept(s) includes a method of decreasing toxic retinoid by-product accumulation within at least one retinal cell. Non-limiting examples of toxic retinoid by-products include, but are not limited to, lipofuscin and bis-retinoids such as but not limited to, N-retinylidene-N-retinyl-ethanolamine (A2E), A2PE, and all-trans-retinal dimers.

In particular embodiments of the methods disclosed herein, administration of the pharmaceutical composition to the subject inhibits rhodopsin regeneration by 40% to 90% with substantially no inhibition of cone function. In additional embodiments, the ophthalmic condition is associated with the deposition of lipofuscin in RPE cells, and wherein administration of the pharmaceutical composition to the subject reduces lipofuscin in at least one RPE cell of the subject. In further additional embodiments, the ophthalmic condition is associated with deposition of A2E in RPE cells, and wherein administration of the pharmaceutical composition to the subject reduces A2E in at least one RPE cell of the subject. In yet further embodiments, administration of the pharmaceutical composition to the subject protects retinal function of the subject.

In any of the methods described herein above, the administration of composition(s) in accordance with the presently disclosed and claimed inventive concept(s) may result in a decrease in expression and/or activity of at least one enzyme in the at least one retinal cell. Thus, an additional embodiment of the presently disclosed and claimed inventive concept(s) includes a method of decreasing expression and/or activity of at least one enzyme in a retinal cell. Non-limiting examples of enzymes so affected include retinal pigment epithelium isomerohydrolases such as retinal pigment epithelium-specific protein 65 kDa (RPE65) isomerohydrolase. In a particular example, the administration of the nitrone composition results in a decrease in RPE65 isomerohydrolase activity.

In any of the methods described herein above, the administration of composition(s) in accordance with the presently disclosed and claimed inventive concept(s) may result in a slowing/inhibition of the Visual Cycle of the at least one retinal cell. Thus, an additional embodiment of the presently disclosed and claimed inventive concept(s) includes a method of slowing/inhibiting the Visual Cycle of a retinal cell. The slowing of the Visual Cycle may include an inhibition of the rate of regeneration of functional rhodopsin without directly affecting the association of 11-cis-retinal with rhodopsin or significantly inhibiting RDH or LRAT activity.

The presently disclosed and claimed inventive concept(s) is further directed to nitrone compositions comprising an inhibitor of RPE65 protein isomerohydrolase activity. Any nitrone composition known in the art or otherwise disclosed herein may be utilized in accordance with the compositions and methods of the presently disclosed and claimed inventive concept(s). For example but not by way of limitation, the nitrone composition may include α-phenyl-tert-butylnitrone (PBN) and/or a PBN derivative/analog.

PBN derivatives/analogs may include at least one substitution at at least one of positions C2, C3, C4, C5 and C6 of the phenyl group. In certain embodiments, the substitution may be a halide, such as but not limited to, a fluoride; particular non-limiting examples of fluoride substitutions include 2-F-PBN, 3-F-PBN, 4-F-PBN, 5-F-PBN, 6-F-PBN, 2,3-di-F-PBN, 2,4-di-F-PBN, 2,5-di-F-PBN, 2,6-di-F-PBN, 3,4-di-F-PBN, 3,5-di-F-PBN, 3,6-di-F-PBN, 4,5-di-F-PBN, 4,6-di-F-PBN, 5,6-di-F-PBN, 2,3,4-tri-F-PBN, 2,3,5-tri-F-PBN, 2,3,6-tri-F-PBN, 2,4,5-tri-F-PBN, 2,4,6-tri-F-PBN, 2,5,6-tri-F-PBN, 3,4,5-tri-F-PBN, 3,4,6-tri-F-PBN, 3,5,6-tri-F-PBN, 4,5,6-tri-F-PBN, 2,3,4,5-tetra-F-PBN, 2,3,4,6-tetra-F-PBN, 2,3,5,6-tetra-F-PBN, 2,3,5,6-tetra-F-PBN, 3,4,5,6-tetra-F-PBN, and 2,3,4,5,6-penta-F-PBN.

In other embodiments, the substitution may include a methyl group; particular non-limiting examples of methyl substitutions include 2-methyl-PBN, 3-methyl-PBN, 4-methyl-PBN, 5-methyl-PBN, 6-methyl-PBN, any di-methyl-PBN (i.e., 2,3-di-methyl-PBN), any tri-methyl-PBN (i.e., 2,3,4-tri-methyl-PBN), any tetra-methyl-PBN (i.e., 2,3,4,5-di-methyl-PBN), and 2,3,4,5,6-penta-methyl-PBN.

In other embodiments, the substitution may include a trifluoromethyl group; particular non-limiting examples of trifluoromethyl substitutions include 2-trifluoromethyl-PBN, 3-trifluoromethyl-PBN, 4-trifluoromethyl-PBN, and 5-trifluoromethyl-PBN.

Additional nitrone compositions that may be utilized in accordance with the methods of the presently disclosed and claimed inventive concept(s) are disclosed in U.S. Pat. No. 5,622,994, issued Apr. 22, 1997 to Carney and Floyd; U.S. Pat. No. 6,002,001, issued Dec. 14, 1999 to Carney and Floyd; and US Patent Application Publication No. US 2010/0168112 A1, published Jul. 1, 2010 to Kelly et al. The entire contents of each of the above-referenced patents and patent applications are hereby expressly incorporated herein.

Additional nitrone compositions that may be utilized in accordance with the presently disclosed and claimed inventive concept(s) include compounds of Formula (I):

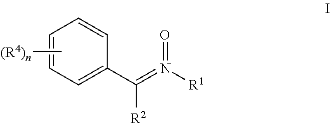

or a pharmaceutically acceptable sale or stereoisomer thereof, wherein R¹ is substituted or unsubstituted alkyl; R² is H, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl; each R⁴ is independently selected from halo, alkyl, haloalkyl, substituted or unsubstituted alkoxy, alkoxyalkyl, cyano, nitro, SO₃H, SOR^{4a}, SO₂R^{4a}, SO₂NR^{4a}R^{4b}; each R^{4a} and R^{4b} is independently H, substituted or unsubstituted alkyl, substituted or unsubstituted aryl, or substituted or unsubstituted heteroaryl; or R^{4a} and R^{4b} taken together with the N they are attached to form heterocycle; and n is 0, 1, 2, 3, 4, or 5.

In certain embodiments, n may be 0. In other embodiments, n may be 1, and R^{4a} may be 2-SO₃H, and R¹ is other than t-Bu. In other embodiments, n is 0, and R¹ is other than t-Bu. In additional embodiments, n may be 1 or 2, and each R⁴ is independently alkyl or haloalkyl. In other embodiments, each R⁴ may independently be Me, Et, i-Pr, n-Pr, n-Bu, i-Bu, t-Bu, or CF₃. In other embodiments, n is 1, 2, 3, 4, or 5. In yet additional embodiments, R¹ is alkyl, Me, Et, i-Pr, n-Pr, n-Bu, i-Bu, or t-Bu. In yet other embodiments, n may be 1 or 2, and R⁴ is independently Cl or F.

In certain embodiments, R² may be H. In additional embodiments, R⁴ may be halo, cyano, methoxy, CF₃, or OCF₃, and R² may be H. In additional embodiments, R⁴ may be halo, cyano, methoxy, CF₃, or OCF₃; R² may be H; and R¹ is other than t-Bu. In further embodiments, n may be 2, and each R⁴ is SO₃H.

In additional embodiments, R⁴ may be SOR^{4a} or SO₂R^{4a}, or R⁴ may be SO₂NR^{4a}R^{4b}. In yet additional embodiments, R^{4b} may be H, alkyl, Me, Et, n-Pr, i-Pr, n-Bu, or t-Bu. In this instance, R^{4a} and R^{4b} taken together with the N they are attached to may form a heterocycle, or may form pyrrolidinyl, piperidinyl, morpholinyl, or piperazinyl. In any of these embodiments, R^{4a} may be H, alkyl, aryl, or heteroaryl, or H, Me, Et, n-Pr, i-Pr, n-Bu, or t-Bu, Ph, or pyridyl.

In further embodiments, R⁴ may be substituted or unsubstituted alkyl, C₁-C₆ alkyl, Me, Et, n-Pr, i-Pr, n-Bu, t-Bu, C₂-C₆ alkoxy, —OMe, —OEt, —O-n-Pr, —O-i-Pr, —O-n-Bu, or —O-t-Bu.

Additional nitrone compositions that may be utilized in accordance with the presently disclosed and claimed inventive concept(s) include compounds of Formula (II):

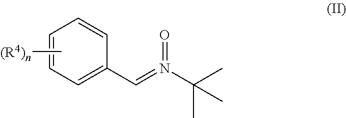

or a pharmaceutically acceptable salt thereof, or a stereoisomer thereof; wherein n is 1, 2, 3, 4, or 5; and R⁴ is as described for Formula (I).

In certain embodiments, n may be 0. In other embodiments, n may be 1 or 2, and each R⁴ is independently alkyl or haloalkyl. In additional embodiments, each R⁴ may independently be Me, Et, i-Pr, n-Pr, n-Bu, i-Bu, t-Bu, or CF₃. In yet further embodiments, n may be 1 or 2, and R⁴ is independently Cl or F.

Additional nitrone compositions that may be utilized in accordance with the presently disclosed and claimed inventive concept(s) include compounds according to formulas (IIIa), (IIIb), (IIIc), (IIId), (IIIe), and (IIIf):

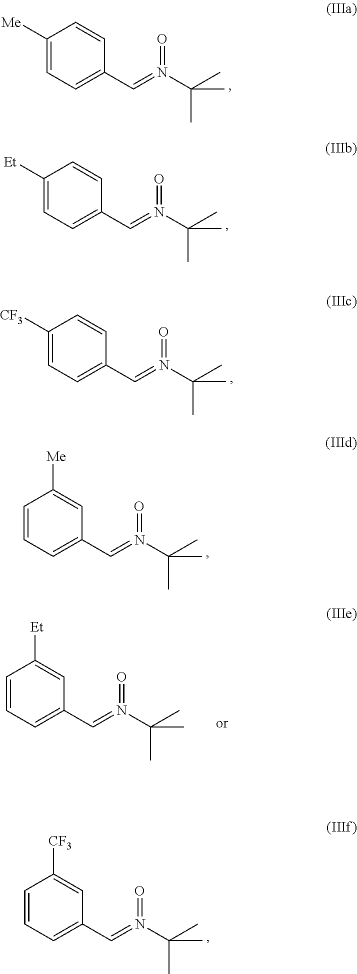 [SMILES: Cc1ccc(/C=[N+](\[O-])C(C)(C)C)cc1 ; CCc1ccc(/C=[N+](\[O-])C(C)(C)C)cc1 ; CC(C)(C)/[N+]([O-])=C/c1ccc(C(F)(F)F)cc1 ; Cc1cccc(/C=[N+](\[O-])C(C)(C)C)c1 ; CCc1cccc(/C=[N+](\[O-])C(C)(C)C)c1 ; CC(C)(C)/[N+]([O-])=C/c1cccc(C(F)(F)F)c1]

or a pharmaceutically acceptable salt thereof, or a stereoisomer thereof.

In one embodiment, the compound is according to formula (IIIa). In another embodiment, the compound is according to formula (IIIb). In yet another embodiment, the compound is according to formula (IIIc). In yet another embodiment, the compound is according to formula (IIId). In yet another embodiment, the compound is according to formula (IIIe). In yet another embodiment, the compound is according to formula (IIIf).

In one embodiment, with respect to the method, the compound is according to formula (I) or (II), n is 1, 2, 3, 4, or 5; and each R⁴ is independently halo. In another embodiment, each R⁴ is independently Cl or F. In a particular embodiment, each R⁴ is independently F. In another particular embodiment, n is 1, and R⁴ is Cl. In yet another particular embodiment, n is 1, and R⁴ is F. In another particular embodiment, n is 2, and each R⁴ is Cl. In yet another particular embodiment, n is 2, and each R⁴ is F. In another particular embodiment, n is 3, and each R⁴ is Cl. In yet another particular embodiment, n is 3, and each R⁴ is F. In another particular embodiment, n is 4, and each R⁴ is Cl. In yet another particular embodiment, n is 4, and each R⁴ is F. In another particular embodiment, n is 5, and each R⁴ is Cl. In yet another particular embodiment, n is 5, and each R⁴ is F. In yet another embodiment, n is 1 or 2, and R⁴ is independently Cl or F.

Additional nitrone compositions that may be utilized in accordance with the presently disclosed and claimed inventive concept(s) include compounds according to formulas (IVa), (IVb), (IVc), (IVd), (IVe), and (IVf):

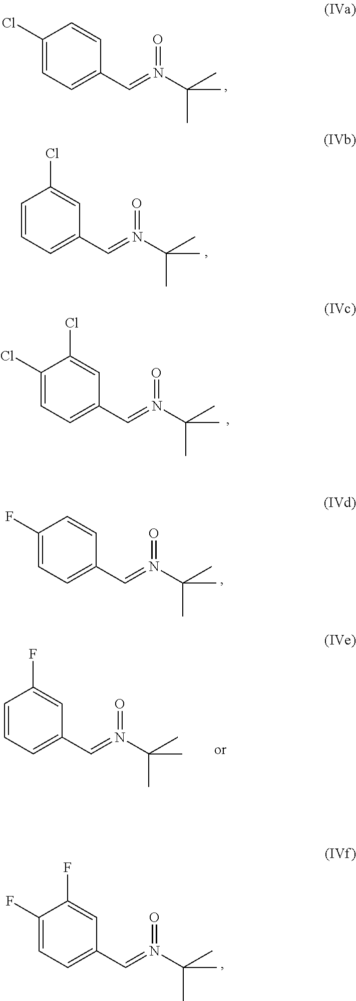

or a pharmaceutically acceptable salt thereof, or a stereoisomer thereof.

In one embodiment, the compound is according to formula (IVa). In another embodiment, the compound is according to formula (IVb). In yet another embodiment, the compound is according to formula (IVc). In yet another embodiment, the compound is according to formula (IVd). In yet another embodiment, the compound is according to formula (IVe). In yet another embodiment, the compound is according to formula (IVf).

In one embodiment, the compound is according to formula (I) or (II), n is 1 or 2; and each R⁴ is independently SO₃H, SOR^{4a}, or SO₂R^{4a}; and R^{4a} is alkyl or aryl. In one embodiment, n is 1, and R⁴ is other than 2-SO₃H. In another embodiment, n is 1, and R⁴ is 3-SO₃H or 4-SO₃H. In another embodiment, n is 1, and R⁴ is 2-SOR^{4a}, or 2-SO₂R^{4a}; and R^{4a} is alkyl or aryl. In another embodiment, n is 2, and R⁴ is 2,4-di-SOR^{4a}, or 2,4-di-SO₂R^{4a}; and R^{4a} is alkyl or aryl. In one embodiment, each R^{4a} is independently Me, Et, i-Pr, n-Pr, n-Bu, i-Bu, t-Bu, or CF₃. In a particular embodiment, each R^{4a} is Me. In another particular embodiment, each R^{4a} is CF₃.

Additional nitrone compositions that may be utilized in accordance with the presently disclosed and claimed inventive concept(s) include compounds according to formulas (Va), (Vb), (Vc), (Vd), (Ve), and (Vf):

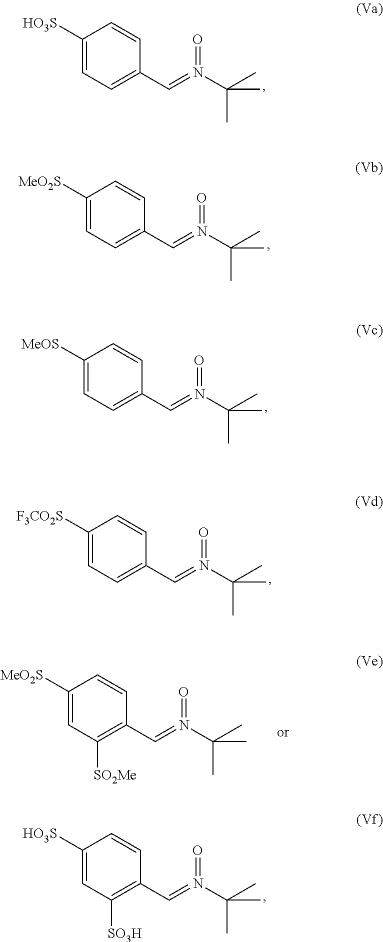

or a pharmaceutically acceptable salt thereof, or a stereoisomer thereof.

In one embodiment, the compound is according to formula (I), the compound is according to formula (Va) or (Vf); and the compound is a pharmaceutically acceptable salt. In one particular embodiment, the compound is sodium, di-sodium, potassium or di-potassium salt.

In one embodiment, the compound is according to formula (Va). In another embodiment, the compound is according to formula (Vb). In yet another embodiment, the compound is according to formula (Vc). In yet another embodiment, the compound is according to formula (Vd). In yet another embodiment, the compound is according to formula (Ve). In yet another embodiment, the compound is according to formula (Vf).

In one embodiment, the compound is according to formula (I) or (II), n is 1 or 2; and each R⁴ is independently SO₂NR^{4a}R^{4b}; each R^{4a} and R^{4b} is independently H, substituted or unsubstituted alkyl. In one embodiment, n is 1, and R⁴ is 2-SO₂NR^{4a}R^{4b}. In one embodiment, n is 1, and R⁴ is 3-SO₂NR^{4a}R^{4b}. In another embodiment, n is 1, and R⁴ is 4-SO₂NR^{4a}R^{4b}. In another embodiment, n is 2, and R⁴ is 2,4-di-SO₂NR^{4a}R^{4b}. In one particular embodiment, each R^{4a} and R^{4b} is independently Me, Et, i-Pr, n-Pr, n-Bu, i-Bu, or t-Bu. In another embodiment, each R^{4a} and R^{4b} is Me. In another particular embodiment, R^{4a} and R^{4b} taken together with the N they are attached to form heterocycle. In another particular embodiment, R^{4a} and R^{4b} along with the N they are attached to form pyrrolidinyl, piperidinyl, morpholinyl, or piperazinyl.

Additional nitrone compositions that may be utilized in accordance with the presently disclosed and claimed inventive concept(s) include compounds according to formulas (VIa), (VIb), (VIc), (VId), and (VIe):

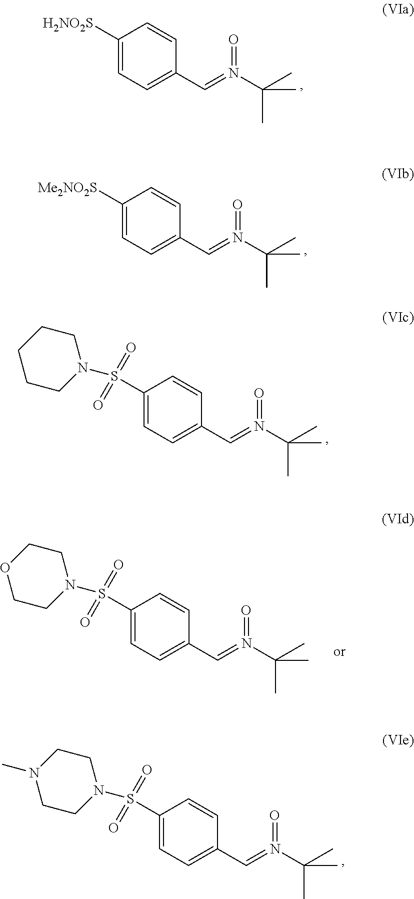

or a pharmaceutically acceptable salt thereof, or a stereoisomer thereof.

In one embodiment, the compound is according to formula (VIa). In another embodiment, the compound is according to formula (VIb). In yet another embodiment, the compound is according to formula (VIc). In yet another embodiment, the compound is according to formula (VId). In yet another embodiment, the compound is according to formula (VIe). In yet another embodiment, the compound is according to formula (VIf).

In one embodiment, the compound is according to formula (I) or (II), n is 1 or 2, and each R⁴ is CN or NO₂. In one embodiment, n is 1, and R⁴ is 2-CN, 3-CN, or 4-CN. In another embodiment, n is 1, and R⁴ is 2-NO₂, 3-NO₂, or 4-NO₂. In another embodiment, n is 2, and R⁴ is 2,4-di-CN. In another embodiment, n is 2, and R⁴ is 2,4-di-NO₂.

In one embodiment, with respect to the method, the compound is according to formula (I) or (II), n is 2 or 3, and one of the R⁴'s is CN, and the remainder are Cl or F.

Additional nitrone compositions that may be utilized in accordance with the presently disclosed and claimed inventive concept(s) include compounds according to formulas (VIIa), (VIIb), (VIIc), (VIId), (VIIe), and (VIIf):

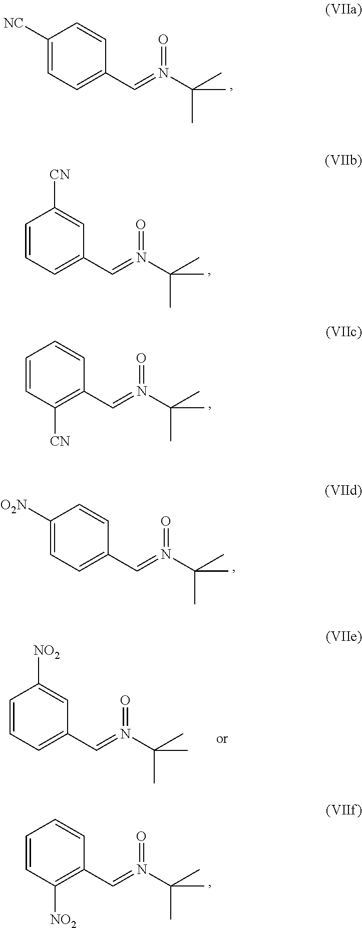 [SMILES: CC(C)(C)/[N+]([O-])=C/c1ccc(C#N)cc1 ; CC(C)(C)/[N+]([O-])=C/c1cccc(C#N)c1 ; CC(C)(C)/[N+]([O-])=C/c1ccccc1C#N ; CC(C)(C)/[N+]([O-])=C/c1ccc([N+](=O)[O-])cc1 ; CC(C)(C)/[N+]([O-])=C/c1cccc([N+](=O)[O-])c1 ; CC(C)(C)/[N+]([O-])=C/c1ccccc1[N+](=O)[O-]]

or a pharmaceutically acceptable salt thereof, or a stereoisomer thereof.

In one embodiment, the compound is according to formula (VIIa). In another embodiment, the compound is according to formula (VIIb). In yet another embodiment, the compound is according to formula (VIIc). In yet another embodiment, the compound is according to formula (VIId). In yet another embodiment, the compound is according to formula (VIIe). In yet another embodiment, the compound is according to formula (VIIf).

The nitrone compositions of the presently disclosed and claimed inventive concept(s) also include pharmaceutically acceptable salts, stereoisomers, solvates, hydrates, prodrugs, tautomers, isotopic variants, or N-oxides of any of the compounds described herein above, as well as combinations thereof.

Additional embodiments of the presently disclosed and claimed inventive concept(s) include pharmaceutical compositions that include an inhibitor of RPE65 protein isomerohydrolase activity in combination with a pharmaceutically acceptable carrier. The inhibitor may be any of the nitrone compositions described herein above. In certain embodiments, the inhibitor may inhibit RPE65 protein isomerohydrolase activity in an RPE cell, and the inhibition of RPE65 protein isomerohydrolase activity may occur in and affect the photoreceptor visual cycle.

In certain embodiments, the formulation of the pharmaceutical compositions comprises water. In another embodiment, the formulation comprises a cyclodextrin derivative. In certain embodiments, the formulation comprises hexapropyl-β-cyclodextrin. In a more particular embodiment, the formulation comprises hexapropyl-β-cyclodextrin (10-50% in water).

The following non-limiting formulation examples illustrate representative pharmaceutical compositions that may be prepared in accordance with the presently disclosed and claimed inventive concept(s). However, any formulations and methods of preparation thereof that are known in the art or otherwise disclosed herein fall within the scope of the presently disclosed and claimed inventive concept(s).

Formulation 1—Tablets: A compound of the presently disclosed and claimed inventive concept(s) is admixed as a dry powder with a dry gelatin binder in an approximate 1:2 weight ratio. A minor amount of magnesium stearate is added as a lubricant. The mixture is formed into 240-270 mg tablets (80-90 mg of active compound per tablet) in a tablet press.

Formulation 2—Capsules: A compound of the presently disclosed and claimed inventive concept(s) is admixed as a dry powder with a starch diluent in an approximate 1:1 weight ratio. The mixture is filled into 250 mg capsules (125 mg of active compound per capsule).

Formulation 3—Liquid: A compound of the presently disclosed and claimed inventive concept(s) (125 mg) is admixed with sucrose (1.75 g) and xanthan gum (4 mg), and the resultant mixture is blended, passed through a No. 10 mesh U.S. sieve, and then mixed with a previously made solution of microcrystalline cellulose and sodium carboxymethyl cellulose (11:89, 50 mg) in water. Sodium benzoate (10 mg), flavor, and color are diluted with water and added with stirring. Sufficient water is then added to produce a total volume of 5 mL.

Formulation 4—Tablets: A compound of the presently disclosed and claimed inventive concept(s) is admixed as a dry powder with a dry gelatin binder in an approximate 1:2 weight ratio. A minor amount of magnesium stearate is added as a lubricant. The mixture is formed into 450-900 mg tablets (150-300 mg of active compound) in a tablet press.

Formulation 5—Injection: A compound of the presently disclosed and claimed inventive concept(s) is dissolved or suspended in a buffered sterile saline injectable aqueous medium to a concentration of approximately 5 mg/mL.

Formulation 6—Tablets: A compound of the presently disclosed and claimed inventive concept(s) is admixed as a dry powder with a dry gelatin binder in an approximate 1:2 weight ratio. A minor amount of magnesium stearate is added as a lubricant. The mixture is formed into 90-150 mg tablets (30-50 mg of active compound per tablet) in a tablet press.

Formulation 7—Tablets: A compound of the presently disclosed and claimed inventive concept(s) is admixed as a dry powder with a dry gelatin binder in an approximate 1:2 weight ratio. A minor amount of magnesium stearate is added as a lubricant. The mixture is formed into 30-90 mg tablets (10-30 mg of active compound per tablet) in a tablet press.

Formulation 8—Tablets: A compound of the presently disclosed and claimed inventive concept(s) is admixed as a dry powder with a dry gelatin binder in an approximate 1:2 weight ratio. A minor amount of magnesium stearate is added as a lubricant. The mixture is formed into 0.3-30 mg tablets (0.1-10 mg of active compound per tablet) in a tablet press.

Formulation 9—Tablets: A compound of the presently disclosed and claimed inventive concept(s) is admixed as a dry powder with a dry gelatin binder in an approximate 1:2 weight ratio. A minor amount of magnesium stearate is added as a lubricant. The mixture is formed into 150-240 mg tablets (50-80 mg of active compound per tablet) in a tablet press.

Formulation 10—Tablets: A compound of the presently disclosed and claimed inventive concept(s) is admixed as a dry powder with a dry gelatin binder in an approximate 1:2 weight ratio. A minor amount of magnesium stearate is added as a lubricant. The mixture is formed into 270-450 mg tablets (90-150 mg of active compound per tablet) in a tablet press.

In certain embodiments of use, the compositions of the presently disclosed and claimed inventive concept(s) are administered via injection. Non-limiting exemplary injection dosage levels may range from about 0.1 mg/kg/hour to at least 10 mg/kg/hour, with administration from about 1 to about 120 hours (such as but not limited to, 24 to 96 hours). A preloading bolus of from about 0.1 mg/kg to about 10 mg/kg or more may also be administered to achieve adequate steady state levels. In certain non-limiting examples, the maximum total dose may not be expected to exceed about 2 g/day for a 40 kg to 80 kg human patient.

For the prevention and/or treatment of long-term conditions, such as neurodegenerative and autoimmune conditions, the regimen for treatment may stretch over many months or years; under these conditions, oral administration may be utilized for patient convenience and tolerance. With oral dosing, one to five oral doses per day (such as but not limited to, two to four, and typically three oral doses per day) are representative regimens. Using these dosing patterns, each dose may provide from about 0.01 to about 20 mg/kg of the compound provided herein, with certain doses each providing from about 0.1 to about 10 mg/kg (such as but not limited to, about 1 to about 5 mg/kg).

The presently disclosed and claimed inventive concept(s) is further directed to an ophthalmic composition for topical application to an eye of a subject. The ophthalmic composition comprises a pharmaceutically acceptable carrier and an inhibitor of RPE65 protein isomerohydrolase activity. The inhibitor may be any of the nitrone compositions described herein above. The ophthalmic composition may be provided in any formulation that allows the ophthalmic composition to function in accordance with the presently disclosed and claimed inventive concept(s); for example but not by way of limitation, the ophthalmic composition may be provided in the form of a solution, drops, mist/spray, plasters and pressure sensitive adhesives, ointment, lotion, cream, gel, lyophilized/spray-dried forms, and the like. In one particular embodiment, the ophthalmic composition is provided in a form for topical application, such as but not limited to, an eye drop.

The inhibitor of RPE65 protein isomerohydrolase activity may be present in any of the pharmaceutical/ophthalmic compositions of the presently disclosed and claimed inventive concept(s) at any concentration that allows the pharmaceutical/ophthalmic composition to function in accordance with the presently disclosed and claimed inventive concept(s); for example but not by way of limitation, the inhibitor may be present in a range having a lower level selected from 0.0001%, 0.005%, 0.001%, 0.005%, 0.01%, 0.05%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9% and 2.0%; and an upper level selected from 3%, 3.5%, 4%, 4.5%, 5%, 5.5%, 6%, 6.5%, 7%, 7.5%, 8%, 8.5%, 9%, 9.5%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90% and 95%. Non-limiting examples of particular ranges include a range of from about 0.0001% to about 95%, a range of from about 0.001% to about 75%; a range of from about 0.005% to about 50%; a range of from about 0.01% to about 40%; a range of from about 0.05% to about 35%; a range of from about 0.1% to about 30%; a range of from about 0.1% to about 25%; a range of from about 0.1% to about 20%; a range of from about 1% to about 15%; a range of from about 2% to about 12%; a range of from about 5% to about 10%; and the like.

The pharmaceutically acceptable carrier of the pharmaceutical/ophthalmic compositions described and claimed herein may comprise at least one delivery agent that assists in delivery of the inhibitor to a desired site of delivery; for example but not by way of limitation, at least one delivery agent may be included in an ophthalmic composition to assist in the penetration of a surface of an eye; in certain embodiments, the delivery agent may assist in delivery to a cornea and/or retina of the eye. For example, in order for a topical application to be effective, the composition may need to be able to penetrate the surface of the eye so that it can travel to the desired tissue. This may include penetrating the conjunctiva and/or the cornea. Also, the penetration rate must be sufficient to impart an effective dose. Many drugs do not possess a requisite penetration ability with regard to the tissues of the eye. It should be noted that the external layers of the eye are quite different from the tissues encountered in the stomach and intestinal tract. Thus, while a certain drug may be readily absorbed in the intestines and introduced into the blood supply for systemic administration, the same drug may be incapable of being absorbed by or passing through the substantially avascular outer layers of the conjunctiva or cornea at a minimally acceptable therapeutic concentration. The mechanism of transport or uptake of the drug is entirely different for topical administration than for oral administration.

In certain embodiments, the pharmaceutically acceptable carrier of the pharmaceutical/ophthalmic compositions disclosed and claimed herein may comprise a solubilizer, such as but not limited to, 2-pyrrolidone, N-methyl-2-pyrrolidone (NMP), dimethylformamide, dimethylacetamide, and combinations thereof. In certain embodiments, the pharmaceutically acceptable carrier includes a phosphate buffer. Non-limiting examples of vehicles that may be utilized as pharmaceutically acceptable carriers include: (a) 10 mM potassium borate/bicarbonate buffer (pH 7.2), glycerine, 1.0%, carboxymethylcellulose 0.3%, benzyl alcohol 0.3%, and 30% 1-methyl-2-pyrrolidinone; and (b) 137 mM sodium chloride, 2.7 mM potassium chloride in 10 mM phosphate buffer (pH 7.4), and 30% 1-methyl-2-pyrrolidinone.

For any of the compositions and methods of the presently disclosed and claimed inventive concept(s), the nitrone compounds provided herein can be administered as the sole active agent, or they can be administered in combination with other agents, including other active amines and derivatives. Administration in combination can proceed by any technique apparent to those of ordinary skill in the art including, for example, separate, sequential, concurrent and alternating administration.

The presently disclosed and claimed inventive concept(s) is further directed to a kit that contains one or more of the pharmaceutical compositions/ophthalmic compositions described herein above. The kit may further contain a second agent as described above. If the composition present in the kit is not provided in the form in which it is to be delivered, the kit may further contain a pharmaceutically acceptable carrier or other agent for mixing with the nitrone composition for preparation of the pharmaceutical composition/ophthalmic composition. The kit may also include instructions packaged with the reagents for administration and/or dosing of the compositions contained in the kit. The instructions may be fixed in any tangible medium, such as printed paper, or a computer-readable magnetic or optical medium, or instructions to reference a remote computer data source such as a worldwide web page accessible via the internet.

### EXAMPLES

Examples are provided hereinbelow. However, the presently disclosed and claimed inventive concept(s) is to be understood to not be limited in its application to the specific experimentation, results and laboratory procedures. Rather, the Examples are simply provided as one of various embodiments and are meant to be exemplary, not exhaustive.

### Example 1

### General Synthetic Procedures & Synthesis of Representative Compounds

The compounds provided herein can be purchased or prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one of ordinary skill in the art utilizing routine optimization procedures.

Additionally, as will be apparent to those of ordinary skill in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

The compounds provided herein may be isolated and purified by known standard procedures. Such procedures include (but are not limited to) recrystallization, column chromatography, or HPLC. The following schemes are presented with details as to the preparation of representative substituted biarylamides that have been listed herein. The compounds provided herein may be prepared from known or commercially available starting materials and reagents by one of ordinary skill in the art of organic synthesis.

The enantiomerically pure compounds provided herein may be prepared according to any techniques known to those of ordinary skill in the art. For instance, they may be prepared by chiral or asymmetric synthesis from a suitable optically pure precursor or obtained from a racemate by any conventional technique, for example, by chromatographic resolution using a chiral column or TLC, or by the preparation of diastereoisomers, separation thereof and regeneration of the desired enantiomer. See, e.g., “Enantiomers, Racemates and Resolutions,” by J. Jacques, A. Collet, and S. H. Wilen, (Wiley-Interscience, New York, 1981); S. H. Wilen, A. Collet, and J. Jacques, Tetrahedron, 2725 (1977); E. L. Eliel Stereochemistry of Carbon Compounds (McGraw-Hill, NY, 1962); and S. H. Wilen Tables of Resolving Agents and Optical Resolutions 268 (E. L. Eliel ed., Univ. of Notre Dame Press, Notre Dame, Ind., 1972, Stereochemistry of Organic Compounds, Ernest L. Eliel, Samuel H. Wilen and Lewis N. Manda (1994 John Wiley & Sons, Inc.), and Stereoselective Synthesis A Practical Approach, Mihály Nógrádi (1995 VCH Publishers, Inc., NY, N.Y.).

In certain embodiments, an enantiomerically pure compound of formula (I) may be obtained by reaction of the racemate with a suitable optically active acid or base. Suitable acids or bases include those described in Bighley et al., 1995, Salt Forms of Drugs and Adsorption, in Encyclopedia of Pharmaceutical Technology, vol. 13, Swarbrick & Boylan, eds., Marcel Dekker, New York; ten Hoeve & H. Wynberg, 1985, Journal of Organic Chemistry 50:4508-4514; Dale & Mosher, 1973, J. Am. Chem. Soc. 95:512; and CRC Handbook of Optical Resolution via Diastereomeric Salt Formation, the entire contents of which are expressly incorporated herein by reference.

Enantiomerically pure compounds can also be recovered either from the crystallized diastereomer or from the mother liquor, depending on the solubility properties of the particular acid resolving agent employed and the particular acid enantiomer used. The identity and optical purity of the particular compound so recovered can be determined by polarimetry or other analytical methods known in the art. The diastereoisomers can then be separated, for example, by chromatography or fractional crystallization, and the desired enantiomer regenerated by treatment with an appropriate base or acid. The other enantiomer may be obtained from the racemate in a similar manner or worked up from the liquors of the first separation.

In certain embodiments, enantiomerically pure compound can be separated from racemic compound by chiral chromatography. Various chiral columns and eluents for use in the separation of the enantiomers are available, and suitable conditions for the separation can be empirically determined by methods known to one of ordinary skill in the art. Exemplary chiral columns available for use in the separation of the enantiomers provided herein include, but are not limited to, CHIRALCEL® OB, CHIRALCEL® OB-H, CHIRALCEL® OD, CHIRALCEL® OD-H, CHIRALCEL® OF, CHIRALCEL® OG, CHIRALCEL® OJ and CHIRALCEL® OK chromatography columns (Chiral Technologies Europe SAS, Cedex France).

General processes for preparing compounds of formula (I) are provided as further embodiments of the presently disclosed and claimed inventive concept(s) and are illustrated in Scheme 1.

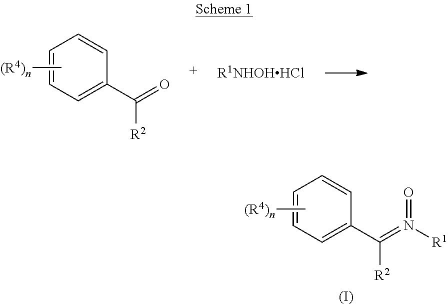

The syntheses of representative compounds of the presently disclosed and claimed inventive concept(s) can be carried out in accordance with the synthetic scheme set forth above, or following the methods described in literature or otherwise known in the art, and using the appropriate reagents, starting materials, and purification methods known to those of ordinary skill in the art. Preparations of representative nitrones of the presently disclosed and claimed inventive concept(s) are provided herein below.

### Compound 1: PBN

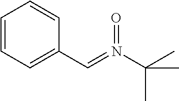

Compound 1 (PBN) was purchased from Sigma-Aldrich (St. Louis, Mo.).

### Compound 2: 4-Fluorophenyl t-butyl nitrone (4-F-PBN)

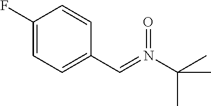

Compound 2 (4-F-PBN) was prepared by following one of the methods described below.

Method A: 4-Fluorobenzaldehyde (8 mmol) and N-tert-butylhydroxylamine (120 mmol) were mixed in chloroform with molecular sieves (50 g, 4 A) and silica gel (10 g). The mixture was sealed under argon gas and stirred for 70 h at room temperature. The mixture was then filtered, and the solid washed with ethyl acetate, and the combined solution was rotary evaporated to give the crude product. The crude product can be purified using HPLC, or other purification methods known to those of ordinary skill in the art.

Method B: A mixture of 4-fluorobenzaldehyde (4 mmol), N-tert-butylhydroxylamine (6 mmol) and p-toluenesulfonic acid in toluene (200 mL) was refluxed for 72 hr under argon gas with a Dean Stark trap to remove generated water. The solution was rotary evaporated to give the crude product. The crude product can be purified using HPLC, or other purification methods known to those of ordinary skill in the art.

Method C: A mixture of tert-buytlhydroxylamine acetate (1.31 g, 8.8 mmoles) and 4-(trifluoromethyl)benzaldehyde (1.00 ml, 7.3 mmoles) in methanol (15 mL) was heated to 100° C. in a sealed tube for 16 hours. The reaction mixture was cooled to room temperature and concentrated to leave an oil. The residue was purified over silica gel by flash chromatography (0 to 100% ethyl acetate/hexane gradient) to give 4-fluorophenyl t-butyl nitrone as a white solid after drying (0.24 grams).

¹H NMR (CDCl₃, 300 MHz) δ 8.30-8.35 (m, 2H), 7.52 (s, 1H), 7.05-7.18 (m, 2H), 1.62 (s, 9H).

MS m/z: 195.9 [M+1]⁺, MW (Calcd) 195.24.

### Compound 3: 4-Trifluoromethylphenyl t-butyl nitrone (4-CF₃-PBN)

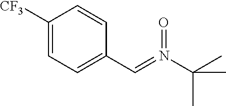 [SMILES: CC(C)(C)/[N+]([O-])=C/c1ccc(C(F)(F)F)cc1]

Compound 3 (4-CF₃-PBN) was prepared by the reaction of 4-trifluoromethylbenzaldehyde with t-butylhydroxylamine and following Method C described for preparation of Compound 2.

¹H NMR (DMSO-d6, 300 MHz) δ 8.45-8.58 (m, 2H), 8.01 (s, 1H), 7.71-7.78 (m, 2H), 1.52 (s, 9H).

MS m/z: 245.9 [M+1]⁺, MW (Calcd) 245.25.

### Compound 4: 4-Methylphenyl t-butyl nitrone (4-Me-PBN)

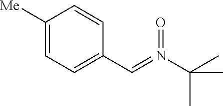

Compound 4 (4-Me-PBN) was prepared by the reaction of 4-methylbenzaldehyde with t-butylhydroxylamine and following Method C described for preparation of Compound 2.

¹H NMR (CDCl₃, 300 MHz) δ 8-11-8.22 (m, 2H), 7.49 (s, 1H), 7.19-7.26 (m, 2H), 2.39 (s, 3H), 1.62 (s, 9H).

MS m/z: 191.9 [M+1]⁺, MW (Calcd) 191.28.

### Compound 5: 4-Methoxyphenyl t-butyl nitrone (4-CH₃O-PBN)

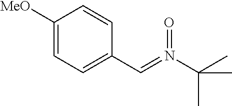

Compound 5 (4-CH₃O-PBN) was prepared by the reaction of 4-methoxybenzaldehyde with t-butylhydroxylamine and following Method C described for preparation of Compound 2.

¹H NMR (CDCl³, 300 MHz) δ 8-24-8.32 (m, 2H), 7.46 (s, 1H), 6.89-6.95 (m, 2H), 3.86 (s, 3H), 1.61 (s, 9H).

MS m/z: 207.9 [M+1]⁺, MW (Calcd) 207.27.

### Compound 6: 4-Ethoxyphenyl t-butyl nitrone (4-EtO-PBN)

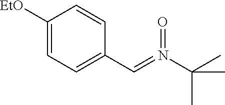 [SMILES: CCOc1ccc(/C=[N+](\[O-])C(C)(C)C)cc1]

Compound 6 (4-EtO-PBN) was prepared by the reaction of 4-ethoxybenzaldehyde with t-butylhydroxylamine and following Method C described for preparation of Compound 2.

¹H NMR (DMSO-d6, 300 MHz) δ 8-26-8.33 (m, 2H), 7.71 (s, 1H), 6.89-6.96 (m, 2H), 4.00-4.02 (m, 2H), (s, 9H), 1.28-1.34 (m, 3H).

MS m/z: 222.0 [M+1]⁺, MW (Calcd) 221.30.

### Compound 7: 2-Fluorophenyl t-butyl nitrone (2-F-PBN)

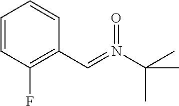

Compound 7 (2-F-PBN) is prepared by the reaction of 2-fluorobenzaldehyde with t-butylhydroxylamine and following Method A, B, or C described for preparation of Compound 2.

### Compound 8: 3-Fluorophenyl t-butyl nitrone (3-F-PBN)

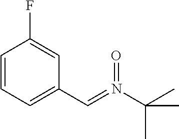

Compound 8 (3-F-PBN) is prepared by the reaction of 3-fluorobenzaldehyde with t-butylhydroxylamine and following Method A or Method B described for preparation of Compound 2.

### Compound 9: 3,5-Difluorophenyl t-butyl nitrone (3,5-diF-PBN)

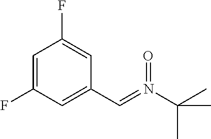

Compound 9 (3,5-di-F-PBN) is prepared by the reaction of 3,5-difluorobenzaldehyde with t-butylhydroxylamine and following Method A, B, or C described for preparation of Compound 2.

### Compound 10: 3,4-Difluorophenyl t-butyl nitrone (3,4-diF-PBN)

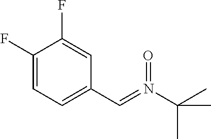

Compound 10 (3,4-di-F-PBN) is prepared by the reaction of 3,4-difluorobenzaldehyde with t-butylhydroxylamine and following Method A, B, or C described for preparation of Compound 2.

### Compound 11: N-tert-butyl-α-(2-sulfophenyl)nitrone (S-PBN)

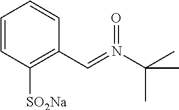

Compound 11 (S-PBN) was purchased from Sigma-Aldrich (St. Louis, Mo.).

### Example 2

The inventors have previously shown that α-phenyl-N-tert-butyl nitrone (PBN, Compound 1), a commonly used free radical spin trap, provides remarkable protection of photoreceptor and RPE cells from light-induced damage (1-5). Because of its anti-oxidant properties and based on several reports demonstrating beneficial pharmacological effects, including reduction in mortality associated with endotoxin shock (6-8), neuroprotection in ischemia-reperfusion and aging models (9-10), and prevention of streptozotocin-induced diabetes in mice (11), the inventors speculated that PBN could be a useful therapeutic intervention against retinal degenerative diseases, such as but not limited to, age-related macular degeneration (AMD). Because the retina has a high oxygen demand, is chronically exposed to light, and contains several photosensitizers, oxidative stress is presently considered to be a cause of disease progression in AMD (12). Thus, the inventors speculated that PBN, which is already known to be effective against age-related and accumulative oxidative stress (13-14), might also be effective against AMD.

The mechanism(s) underlying the PBN-mediated protection of photoreceptor cells are not well understood. Given the role of oxidative stress in retinal light damage (15-18) and the free radical scavenging properties of PBN, the inventors considered that PBN might exert an antioxidant function in the retina by quenching reactive oxygen species generated in the early stages of light-stress. However, besides free radical scavenging, PBN has a multitude of pharmacological effects in neuroprotection, such as modulating the expression of various cytokine genes, such as but not limited to, Cox-2, iNos and the transcription factors NF-κB and AP-1 (19-20). As AP-1 is involved in light-induced photoreceptor cell death (21-22), inhibition of c-fos activation has been proposed as one of the mechanisms of PBN-mediated protection of photoreceptors (5). However, the mechanism of c-fos gene down regulation by PBN is not clear. As a functional rhodopsin visual pigment is necessary to elicit the damaging effects of light (23), the inventors speculated that PBN might influence the rate of visual pigment regeneration by slowing the retinoid visual cycle.

The retinoid visual cycle is a multi-step process for the recycling of 11-cis-retinal (the chromophore of both rod and cone visual pigments) and is essential for regeneration of visual pigment and maintenance of normal vision. The key step of the visual cycle is the hydrolysis-isomerization of all-trans-retinyl ester to 11-cis-retinol. Previously, it has been established that the enzyme that catalyzes the isomerization step is RPE65 isomerohydrolase, which is expressed in retinal pigment epithelial cells (24-26). Intact RPE65 function is essential for vision, as mutations in the RPE65 gene have been reported to cause several forms of inherited retinal dystrophies (27-31). Moreover, no 11-cis-retinoids were detected in the Rpe65^{-/-} mice, suggesting that RPE65 is the only enzyme that can produce 11-cis-retinoids in the eye (32-33). Rpe65^{-/-} mice were found to be completely protected against light induced apoptosis (34). In this Example, it is established that RPE65 isomerohydrolase activity is efficiently inhibited by PBN slowing down the visual pigment regeneration rate.

### Experimental Procedures of Example 2

Animal care—All procedures were performed according to the ARVO Statement for the Use of Animals in Ophthalmic and Vision Research and the University of Oklahoma Health Sciences Center (OUHSC) Guidelines for Animals in Research. All protocols were reviewed and approved by the Institutional Animal Care and Use Committees of the OUHSC and the Dean A. McGee Eye Institute (DMEI). Albino Sprague Dawley (SD) rats and BALB/c mice were born and raised in the DMEI vivarium and maintained from birth under dim cyclic light (5 lux, 12 hours on/off, 7 AM-7 PM central time).

PBN treatment and light-exposure regimens—PBN is a lipophilic compound that is sparsely soluble in aqueous solvents [solubility is up to 15 mg/mL in phosphate-buffered saline (PBS)]. N-tert-butyl-α-(2-sulfophenyl)nitrone sodium salt (S-PBN) is much more soluble in water (Compound 1) and also possesses similar free radical trapping properties (35). Consistent with previous publications (1-2,5), a dose of 50 mg/kg PBN and S-PBN dissolved in saline was chosen for intraperitoneal injection in the rats in the treatment group. An equal volume of saline was injected into control rats.

PBN injection in dark-adapted rats 0.5 hour before damaging light exposure provided almost complete protection of the retina from light-induced damage (1-2,5). A light-stress paradigm of 2,700 lux for 6 hours was used to test the effect of PBN at different time points before (0.5 hour, 3 hours, 6 hours, 12 hours, 16 hours, and 24 hours) or after (0.5 hour and 3 hours) light exposure. The effect of S-PBN was tested using the same dose and protocol of PBN at 0.5 hours before light exposure.

Electroretinography—After light exposure, rats were returned to their dim cyclic light (5-10 lux) environment for 7 days before ERG recordings were performed. Flash ERGs were recorded with the Diagnosys Espion E2 ERG System (Diagnosys, LLC, Lowell, Mass.). Rats were maintained in total darkness overnight and prepared for ERG recording under dim red light. They were anesthetized with ketamine (120 mg/kg body weight) and xylazine (6 mg/kg body weight) intramuscularly (IM). One drop of 10% (v/v) phenylephrine was applied to the cornea to dilate the pupil, and one drop of 0.5% (v/v) proparacaine HCl was applied for local anesthesia. Rats were kept on a heating pad at 37° C. during recordings. A gold electrode was placed on the cornea, a reference electrode was positioned in the mouth, and a ground electrode was placed on the foot; rats were then placed inside a Ganzfeld illuminating sphere. Responses were differentially amplified, averaged, and stored. For the assessment of rod photoreceptor function (scotopic ERG), five strobe flash stimuli were presented at flash intensities at −2.3, −1.3, 0.7, and 2.7 log cd·s/m². The amplitude of the a-wave was measured from the pre-stimulus baseline to the a-wave trough. The amplitude of the b-wave was measured from the trough of the a-wave to the peak of the b-wave. For the evaluation of cone function (photopic ERG), a strobe flash stimulus (3.3 log cd·s/m²) was presented to dilated, light-adapted (5 minutes at 1.7 log cd·s/m²) rats. The amplitude of the cone b-wave was measured from the trough of the a-wave to the peak of the b-wave.

Possible defects in the visual cycle were analyzed by measuring the time course of dark adaptation (recovery of rod photoreceptor sensitivity) following a bleaching light exposure. Rats were fully dark-adapted and injected intraperitoneally with PBN (50 mg/kg) or saline. Full-field scotopic ERGs for both eyes were recorded after 3 hours of PBN injection. A single test flash of 2.3 log cd·s/m² was presented to elicit the rod a-wave response under fully dark-adapted conditions. Rats were then exposed to a steady field of log 2.3 cd·s/m² for 2 minutes in the Ganzfeld dome to bleach rod photoreceptors. Immediately following the bleaching period (time=0 minutes) and every 10 minutes thereafter (time=10, 20, 30, 40, 50, and 60 minutes), the same test flash of 2.3 log cd·s/m² was presented. The a-wave responses at the indicated times after bleaching were normalized to the initial dark-adapted response for each rat.

To evaluate cone function under conditions in which rod recovery is suppressed, rats were injected with PBN (50 mg/kg) or saline 60 minutes prior to recording single-flash photopic and flicker-flash ERG recordings. Rats were placed under a steady adapting field of 1.7 log cd·s/m² for at least 7 minutes. A single flash of 3.3 log cd·s/m² was presented under the same adapting field to elicit a maximal cone response. Cone responses were further evaluated by presenting 1.2 log cd·s/m² stimuli flickering at frequencies of 3-, 10-, 20-, and 30-Hz under the same adapting field.

Histology—After ERG recordings, rats were killed by carbon dioxide asphyxiation for light microscopic evaluation of retinal structure. Immediately after death, eyes were excised, placed in fixative (4% (w/v) paraformaldehyde, 2% (v/v) trichloroacetic acid, 20% (v/v) isopropyl alcohol, 2% (v/v) zinc chloride, and 72% (v/v) distilled water), and embedded in paraffin. Sections of 5 μm were cut along the vertical meridian through the optic nerve and stained with hematoxylin and eosin (H&E). The thickness of the outer nuclear layer (ONL) was measured at 0.5 mm distances from the optic nerve to the inferior and superior ora serrata and plotted as a spider-diagram.

Measurement of rhodopsin regeneration—To measure the effect of PBN on the rate of rhodopsin regeneration, dark-adapted rats were injected intraperitoneally with PBN (50 mg/kg) or PBS 0.5 hour prior to a 2 hour bleach in room light (˜400 lux). Immediately following the bleach (time=0 minutes), a group of PBN- and vehicle-treated rats were killed, and retinas were removed and snap frozen. The remaining rats were dark-adapted, and retinas were collected under dim red light at 45, 90, and 180 minutes after the bleach. An additional group of rats was dark-adapted overnight, injected with PBN or vehicle and maintained in darkness for 2 hours to serve as dark-adapted controls and to determine if PBN had a direct effect on the binding of chromophore to rhodopsin.

Rhodopsin measurements were performed as described previously (2,36) with slight modification. Briefly, under dim red light, each retina was homogenized in 450 μl of buffer containing 10 mM Tris-HCl (pH 7.4), 150 mM NaCl, 1 mM EDTA, 2% (w/v) octylglucoside, and 50 mM hydroxylamine. Homogenates were centrifuged at 16,000 g, and soluble lysates were scanned from 270-800 nm in a spectrophotometer (Ultrospec 3000 UV/Vis Spectrophotometer, GE Healthcare, Piscataway, N.J.). Samples were then bleached under room light for 10 minutes and scanned again. The difference spectra at 500 nm between pre- and post-bleached samples were used to determine rhodopsin content using a molar extinction coefficient of 42,000 M⁻¹ (37). The values were normalized to the total lysate volume, and data are presented as rhodopsin content/retina.

Assay for retinal RDH activity—The effect of PBN on retinal dehydrogenase (RDH) enzymes was determined in vitro using mouse retinal microsomes. To prepare retinal microsomes, dissected mouse retinas were homogenized in sucrose buffer [25 mM sucrose, 10 mM Tris-Cl (pH 7.2), and 1 mM EDTA], containing protease inhibitors (2 μg/ml aprotinin, 5 μg/ml pepstatin A, 10 μg/ml leupeptin, and 0.5 mM phenylmethylsulfonyl fluoride, final concentrations), using a Polytron PT-1200 CL (Kinematica Inc, Bohemia, N.Y.). Homogenates were centrifuged at 10,000 g for 10 minutes at 4° C. The resulting supernatants were then centrifuged at 100,000 g for 1 hour at 4° C., and microsomal pellets were resuspended in storage buffer [50 mM Tris-Cl (pH 7.2), 1 mM EDTA, 20% glycerol, 1 mM dithiothreitol, and the protease inhibitors described above]. Protein concentrations were measured, and microsomal fractions were stored at −80° C. after snap freezing in liquid nitrogen.

Reactions were carried out in 1 ml reaction buffer [100 mM Tris-HCl (pH 7.2), 200 mM NaCl, 1 mM dithiothreitol, 2 mg/ml bovine serum albumin, and 1% (v/v) glycerol] with 5 μg of microsomes, 0 or 75 μM of NADPH, 0 or 2 mM PBN, and increasing concentrations of the substrate all-trans-retinal. After 1 hour incubation at room temperature, reactions were terminated by the addition of 1 volume of cold methanol. Retinoids were extracted with 2 volumes of hexane and analyzed by HPLC. Samples were dissolved in HPLC mobile phase (11.2% (v/v) ethyl acetate/2.0% (v/v) dioxane/1.4% (v/v) octanol in hexane), and retinoids were separated by using a LiChrospher® Sil 60 Å 5-μm column (Phenomenex Inc., Torrance, Calif.). The peaks of all-trans-retinal (substrate) and all-trans-retinol (product of the reaction) were identified and quantified by comparison with pure retinoid isomeric standards. All procedures were performed under dim red light. For each condition the non-enzymatic conversion of all-trans-retinal to all-trans-retinol measured in absence of NADPH was subtracted from the conversion obtained with NADPH to determine the NADPH-dependant activity of retinal RDHs.

Isomerohydrolase activity assay and PBN inhibition—Rats were sacrificed, and eyes were enucleated and dissected to remove the anterior part including lens, vitreous, and retina. The remaining eyecups containing RPE were sonicated 3 times for 20 seconds in a cold 0.32 M sucrose/0.1 M sodium phosphate buffer (pH 7.4). The homogenate was centrifuged (20 minutes, 20,000 g) to sediment sclera, unbroken cells, nuclei and mitochondria. The obtained supernatant was recentrifuged (1 hour, 100,000 g), and the microsomal pellet was resuspended in 10 mM 1,3-bis[tris(hydroxymethyl)-methylamino]propane (BTP), (pH 8.0) and 100 mM NaCl, and stored at −80° C. All-trans-[11,12-³H]-retinol (1 mCi/ml, 45.5 Ci/mmol, American Radiolabeled Chemical, Inc., St. Louis, Mo.) in N,N-dimethyl formamide was used as the substrate for the isomerohydrolase assay. For each reaction, 50 μg microsomal proteins from the rat RPE were added into 200 μl of reaction buffer [10 mM BTP (pH 8.0), and 100 mM NaCl] containing 0.2 μM of all-trans-retinol, 1% (w/v) BSA, and 25 μM of cellular retinaldehyde binding protein (CRALBP). For the inhibition studies, PBN or S-PBN dissolved in the PBS (pH 7.4) was added to the reaction prior to addition of all-trans-retinol. The reaction was stopped and retinoids extracted with 300 μl of cold methanol and 300 μl of hexane, and centrifuged at 10,000 g for 5 minutes. The upper layer was collected, and the generated retinoids were analyzed by normal phase HPLC as described (38). The peak of each retinoid isomer was identified based on its characteristic retention time of retinoid standards. The isomerohydrolase activity was calculated from the area of the 11-cis-retinol peak using Radiomatic 610TR software (Perkin Elmer, Boston, Mass.) with synthetic 11-cis-[³H]-retinol as a standard. Alternatively, liposomes composed of 3.3 μM all-trans-retinyl palmitate and 250 μM of a mixture of 1,2-dioleoyl-sn-glycero-3-phosphocholine (DOPC):1,2-dilauroyl-sn-glycero-3-phosphocholine (DLPC) (85:15) were incubated with 125 μg of total protein lysates from 293A cells that had been infected with chicken RPE65 adenovirus. Liposomes and chicken RPE65 were prepared as described previously (39). Non-linear regression analysis of v-versus-[S] data was used to calculate Vₘ (apparent) and Kₘ (apparent) in the absence and in the presence of PBN. The inhibition constant for PBN was calculated from the following equation: Kᵢ=[I]/(Vₘⁱ/Vₘ⁻¹) where [I]=concentration of PBN, Vₘⁱ=maximal velocity in the presence of Vm=maximal velocity in the absence of PBN.

Statistical analyses—Statistical analyses were performed using GraphPad Prism 5.0 software (GraphPad Software, Inc., La Jolla, Calif.). The quantitative data are expressed as mean±SD or SEM for each group. Student's and paired t-tests were performed to assess differences between means.

### Results of Example 2

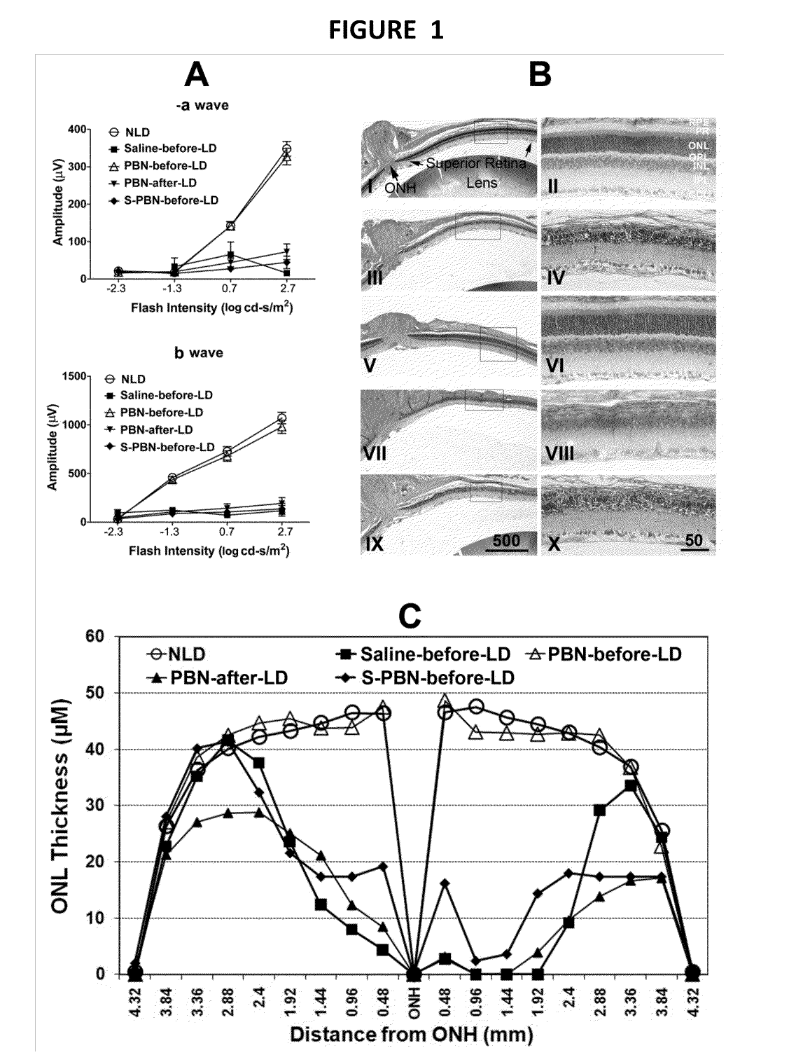

PBN protection of retina from light stress—It has been shown that administration of 50 mg/kg PBN 0.5 hour before light stress (2,700 lux for 24 hours) provided significant (80-90%) protection of retinal structure and function (1). In this Example, the same dose of light was used but for a shorter duration (6 hours); the shorter duration also caused significant loss of photoreceptor cell function (FIG. 1). Electroretinography was performed 7 days after light exposure, and thus the reductions in ERG responses (FIG. 1A) reflect the loss of photoreceptor cells (FIG. 1B). Representative H&E stained sections of retina 7 days after light damage are shown in FIG. 1B. By 7 days, the dead photoreceptors were removed, and the remaining nuclei in the outer nuclear layer (ONL) represent the viable photoreceptors. The findings obtained by functional ERG analysis were confirmed by quantitative morphometry of the thickness of the ONL of the retina which provided a direct indicator of photoreceptor cell number (FIG. 1C). PBN injection 0.5 hour before light exposure almost completely protected against light-induced loss of retinal function (Rod a- and b-wave responses, FIG. 1A) and structure (FIGS. 1B and 1C) [compare NLD (no-light-damage) vs. saline-injected vs. PBN-injected]. Interestingly, the same dose of PBN administered 0.5 hour after light exposure did not provide any protection (FIGS. 1A, 1B, and 1C). Water soluble S-PBN also failed to protect against light-induced loss of photoreceptor structure and function (FIGS. 1A, 1B, and 1C).

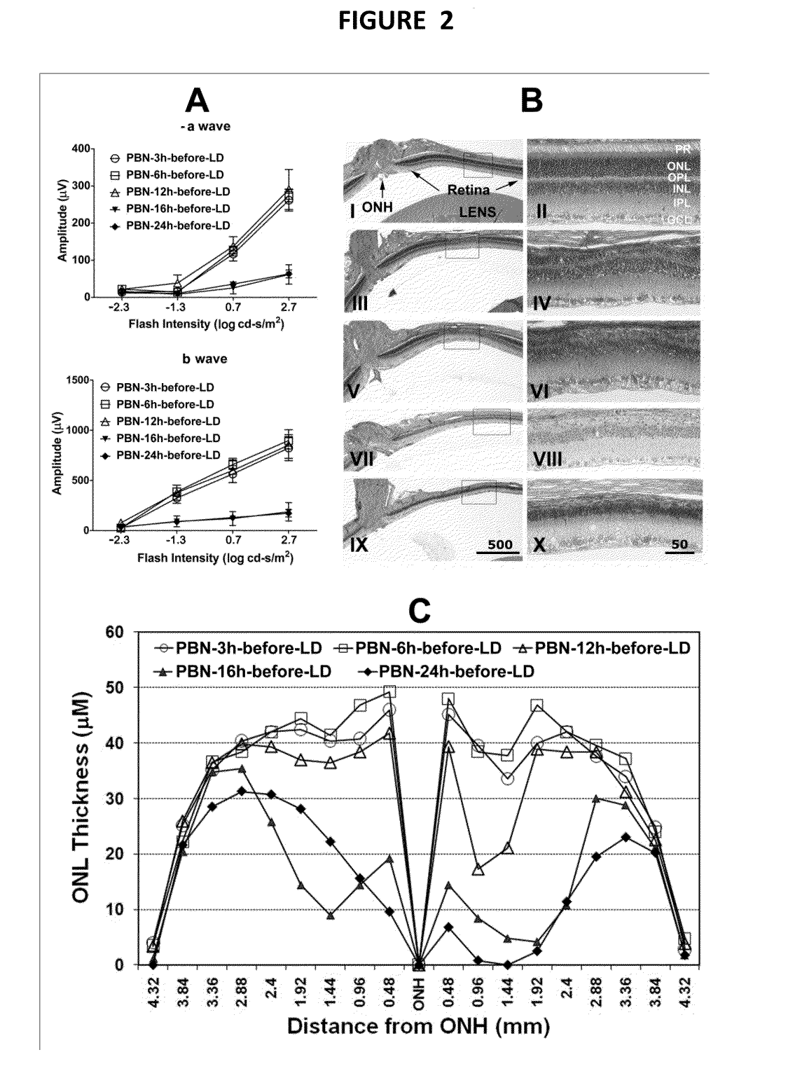

The effect of PBN administration at several time points before light exposure was then evaluated. PBN administration up to 12 hours prior to light exposure protected against light-induced loss of photoreceptor cells, but was not protective when administered 16 or 24 hours before light exposure (FIGS. 2A, 2B and 2C). Although some ONL thinning was observed in a small region of the superior central retina when PBN was administered 6 or 12 hours before damaging light exposure (FIG. 2B), the loss of photoreceptors in this small area was not reflected in the full field ERG responses, which showed complete functional protection (FIG. 2A). The preservation of cone function, as measured by single flash photopic b-wave amplitude 7 days after damaging light exposure, paralleled the results shown for rods. Cone function was preserved in retinas in which PBN was administered between 0.5 hour and 12 hours prior to light exposure. However, cone function was not preserved if PBN was administered 16 or 24 hours before light exposure (FIG. 17). As seen for rods, PBN did not protect cone function when administered after light-damage, and S-PBN failed to protect cone function (FIG. 17). In summary, the results indicate that PBN must be administered within a specific time window to protect effectively, and this protective effect is only effective with the more hydrophobic version of this compound.

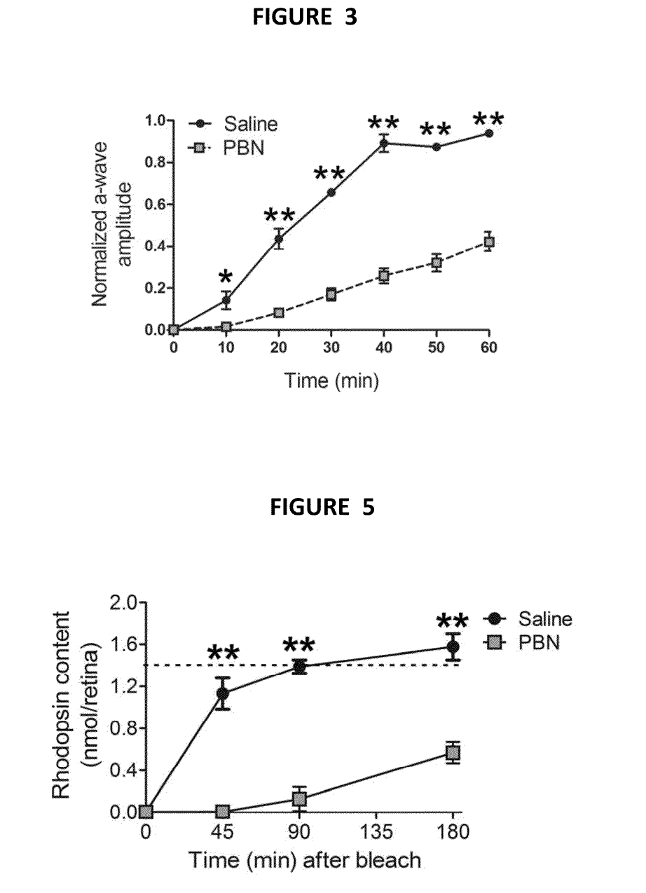

Effect of PBN on the rate of recovery of rod photoresponses: dark adaptation ERG—The damaging effect of bright light requires a functional rhodopsin photopigment (23). It is well established that PBN protects against light damage (1), and the inventors previously speculated that PBN, as part of its protective mechanism, might down-regulate rhodopsin regeneration rate (2). This hypothesis was tested by measuring the in vivo recovery of rod photoresponses following a bleaching illumination as previously described (40-41). After overnight dark-adaptation, rats were injected with PBN or saline 30 minutes prior to exposure to a 2.3 log cd·s/m² test flash to elicit a saturated rod a-wave response. These fully dark-adapted a-wave responses from PBN-treated and control rats were indistinguishable (PBN: −441.5±70.66 μV, saline: −430.9±27.81 μV; n=4), indicating that PBN did not directly affect the rod photocascade when the test flash was presented to fully dark-adapted retinas. However, as shown in FIG. 3, PBN treatment dramatically reduced the rate of recovery of rod photoresponses; 40 minutes after the bleaching illumination, PBN-treated rats had only recovered 25% of the initial fully dark-adapted response, whereas vehicle-treated rats had recovered completely. These results demonstrate that PBN reduces the dark adaptation rate of rods.

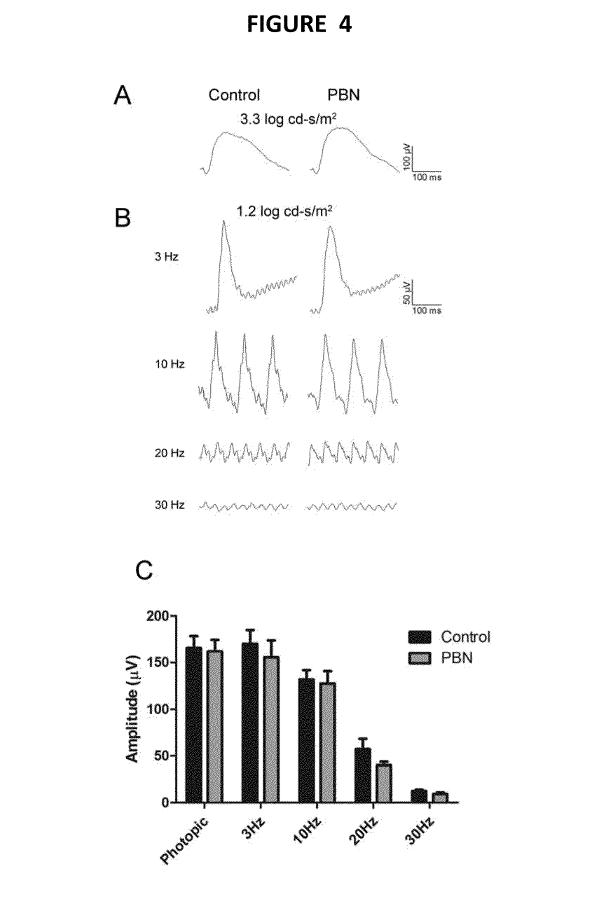

To assess the effect of PBN administration on cone function under conditions when rod recovery was suppressed, single- and flicker-flash ERG recordings were performed under a steady, rod-suppressing background illumination 60 minutes after PBN administration. As shown in FIG. 4, cone function was not affected by PBN under conditions when rod recovery was completely suppressed.

Effect of PBN on rhodopsin regeneration—To test whether the dramatic PBN-induced delay of dark adaptation was mediated by direct inhibition of the rate of regeneration of functional rhodopsin, rhodopsin content was measured spectrophotometrically at several time points before, during, and after a complete photobleach. As indicated in FIG. 5, rhodopsin recovery was significantly inhibited by administration of PBN. PBN was administered to fully (overnight) dark-adapted rats that were subsequently exposed to room light for 2 hours, resulting in a complete bleach of rhodopsin in both saline- and PBN-injected rats (FIG. 5, time=0 minutes). When returned to darkness for 45 minutes, virtually no rhodopsin was regenerated in PBN-treated rats, whereas rhodopsin recovery in controls was already 75% of fully dark-adapted values (dotted line, FIG. 5). Even after dark adaptation for 3 hours, only ˜30% of the bleachable rhodopsin had recovered in PBN treated rats, while saline-injected control levels had fully recovered. These results demonstrate profound inhibition of rhodopsin recovery by PBN administration in vivo. Administration of PBN to rats maintained in darkness did not reduce rhodopsin content [mean±SEM (nmol/eye), PBN=1.38±0.14; saline 1.41±0.07)], indicating that PBN did not directly displace chromophore from the visual pigment.

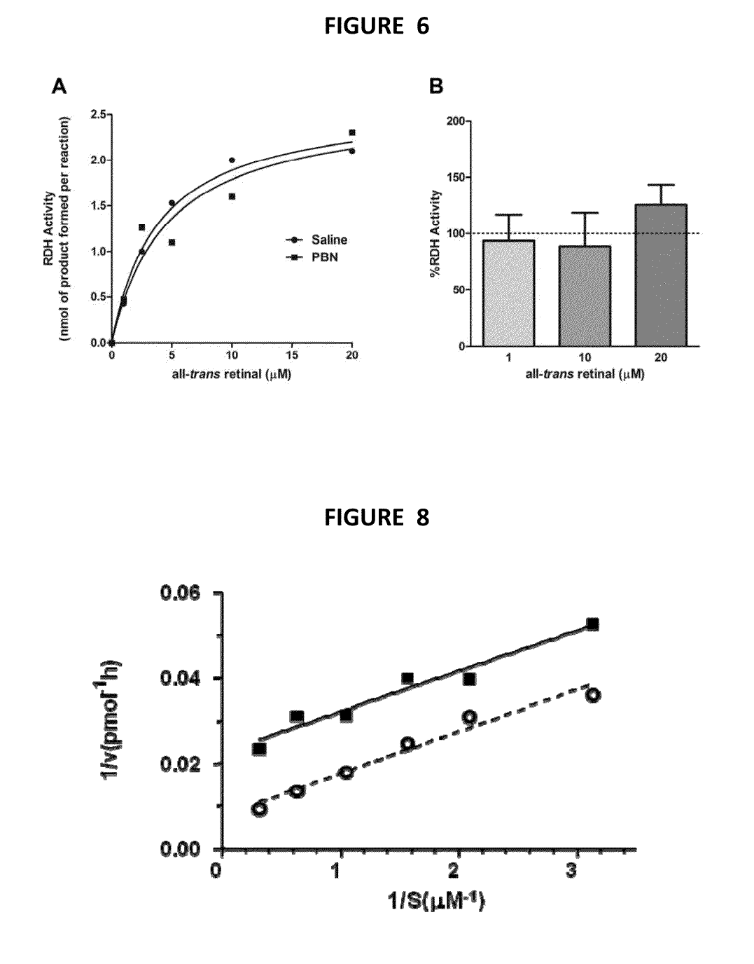

Effect of PBN on retinol dehydrogenase activity—The dark adapted ERG and the rhodopsin regeneration kinetics presented in FIGS. 3 and 5, respectively, indicated that PBN delayed the regeneration of functional visual pigment without directly affecting the association of 11-cis-retinal with rhodopsin. These results suggested that PBN might exert its effects on some enzymes of the visual cycle. To test this, the effect of PBN on the activities of major retinal retinol dehydrogenases (RDHs) and RPE enzymes (LRAT and RPE65) responsible for maintaining the visual cycle was evaluated. Several RDHs are expressed in the retina and participate in the regeneration of the 11-cis-retinal chromophore of rhodopsin by catalyzing the first step of the visual cycle, which is the reduction of all-trans-retinal to all-trans-retinol, using NADPH as coenzyme. To determine if PBN could directly inhibit retinal RDH activity, an in vitro assay with mouse retinal microsome preparation as a source of RDH enzymes was used. NADPH-dependent reduction of all-trans-retinal was measured in the presence and absence of PBN. A representative saturation curve is shown in FIG. 6A. The addition of PBN did not inhibit retinal RDH activity. Results from the average of 3 experiments presented in FIG. 6B show that PBN does not significantly inhibit RDH activity.

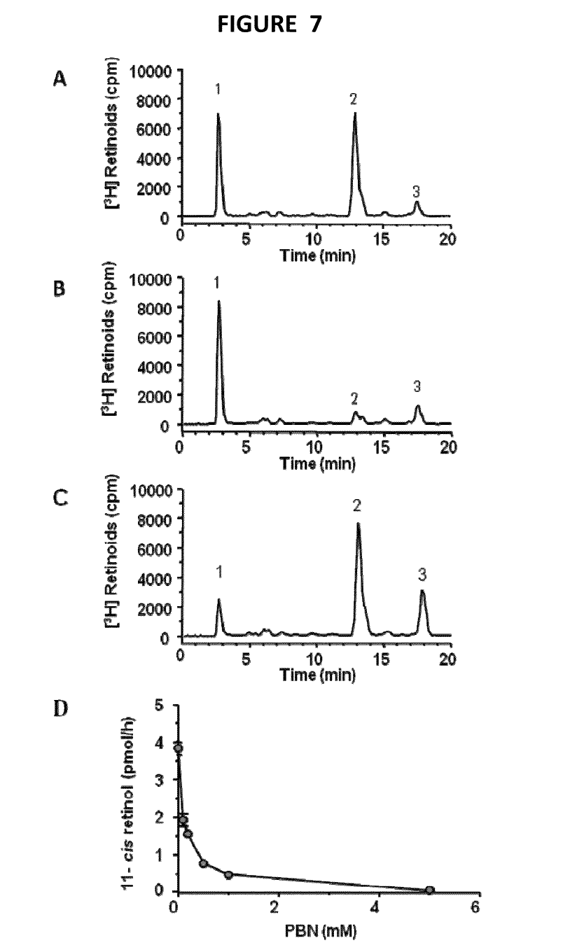

Effect of PBN on RPE65 isomerohydrolase activity—It has been previously shown that conversion of all-trans-retinyl ester to 11-cis-retinol catalyzed by RPE65 is a rate-limiting step in the visual cycle (42). To determine the direct inhibition of PBN on isomerohydrolase activity of RPE65, an in vitro isomerohydrolase assay was used. Although all-trans-retinyl ester is the endogenous substrate for the RPE65 isomerohydrolase (38), the insolubility of retinyl ester in water prevented its direct use in the reaction. Therefore, all-trans-[³H]-retinol was used to produce retinyl esters by LRAT in rat RPE microsomes; these esters were then converted directly to 11-cis-[³H]-retinol by RPE65. Incubation of the rat RPE microsomes with all-trans-[³H]-retinol resulted in formation of all-trans-retinyl esters and significant amounts of 11-cis-[³H]-retinol, as shown by the HPLC elution profile (FIG. 7A). The addition of 1 mM PBN to the reaction resulted in almost complete inhibition of 11-cis-retinol generation (FIG. 7B), whereas the production of retinyl ester did not decrease, suggesting that LRAT was not inhibited by PBN. The PBN inhibition of isomerohydrolase activity was concentration-dependent (FIG. 7D), with an apparent IC₅₀ of 0.1 mM. Interestingly, S-PBN in the same enzymatic assay did not inhibit RPE65 generation of 11-cis-retinol at a concentration as high as mM (FIG. 7C).

To completely exclude the possibility that diminished 11-cis-retinol production could result from PBN-mediated inhibition of LRAT activity and to analyze the model of inhibition, a recently developed liposome-based isomerohydrolase assay (39) was employed. In this assay, a hydrophobic substrate of RPE65, all-trans-retinyl palmitate, was incorporated into DOPC:DLPC liposomes; this molecule can be directly converted to 11-cis-retinol in the isomerohydrolase reaction, allowing the determination of the type of inhibition. In this experiment, recombinant chicken RPE65, which has a more efficient isomerohydrolase activity compared to that from rod-dominant species, was used (43). To determine the type of inhibition, the isomerohydrolase activity at various substrate concentrations was measured in the presence and absence of PBN. As shown in the Lineweaver-Burk graph (FIG. 8), both Vₘ and Kₘ decreased in the presence of PBN, resulting in a plot consisting of two parallel lines characteristic of uncompetitive inhibition. The inhibition constant was calculated according to the formula for uncompetitive inhibition (44), which yields Kᵢ=53±7 μM. These results agree well with the Kᵢ recently reported for PBN (45).

### Discussion of Example 2

Light-induced retinal degeneration is a useful model for retinal research because photoreceptor cell death occurs by apoptosis, as is the case in hereditary retinal degenerations such as retinitis pigmentosa and age-related macular degeneration (46). Because of its advantage of causing synchronized and regulated cell death, this model has been used extensively to screen putative neuroprotective compounds (16, 47-48). Since its discovery in 1966 by Noell et al. (49), a number of studies have identified that photobleaching of rhodopsin is the essential trigger for retinal light damage (34,50). The availability of retinol is also crucial, as vitamin A-deficient rats are protected against light damage (51-52). Rhodopsin knockout (KO) mice, which lack the opsin apoprotein, and RPE-65 KO mice, which have opsin apoprotein but lack the ability to generate 11-cis-retinal, are both protected against light damage (33-34,50).

A steady state rhodopsin level is achieved by the balance between its bleaching and regeneration, which involves both the RPE and photoreceptors. Therefore, the rate of rhodopsin regeneration is an important factor in light damage susceptibility. Fast regeneration of functional rhodopsin after bleaching increases retinal sensitivity to light damage, whereas slowing the flux of retinoids through the visual cycle increases the resistance of photoreceptors to light-induced insult. For example, slowing rhodopsin regeneration and inhibiting the visual cycle with 13-cis-retinoic acid prevents light damage in albino rats (53). 13-cis-retinoic acid was shown to be a competitive inhibitor of RPE65, the rate-limiting enzyme of the visual cycle (54). More recently, it has been shown that the RPE65 inhibitor retinyl amine provides efficient protection from light damage (55). In mice, RPE65 enzymatic activity and the rate of rhodopsin regeneration are related, and slowing regeneration results in resistance to light damage (56). It has been shown that the extent of light damage is significantly lower in a mouse strain with a Leu450Met mutation in RPE65 (56). This mutation decreased the isomerohydrolase activity in RPE by approximately 5-fold (38), making the retina of Met450 mice significantly resistant to light damage (57).

Most of the small molecules inhibiting the visual cycle are structurally similar to retinoids. Although PBN (Compound 1) does not have structural similarity to RPE65 substrates, its effect was nevertheless analyzed on the visual cycle enzymes such as RDH and RPE65. Given the profound protective effect of PBN against light-induced retinal degeneration, it was hypothesized that PBN may interfere with rhodopsin regeneration during the continuous illumination in the light damage animal model. This would be predicted to desensitize the retina to the damaging light. Based on the observation that PBN significantly slowed the rate of dark adaptation after bleaching (FIGS. 3 and 5), the mechanism could be one of the following: 1) PBN may interfere directly with the binding of 11-cis-retinal chromophore to opsin, or 2) PBN may inhibit one or more of the enzymes of the visual cycle involved in the generation of 11-cis-retinal chromophore. The results of this Example illustrate the latter mechanism. It was observed that PBN did not have any effect on opsin-chromophore interaction or on phototransduction in the dark (FIGS. 3 and 5). Therefore, the direct effect of PBN on the enzymatic activities of visual cycle RDHs, LRAT, and RPE65 was evaluated. It was observed that PBN did not affect the activity of RDHs or LRAT (FIG. 6) but significantly inhibited RPE65 activity (FIGS. 7 and 8). RPE65 is the only isomerohydrolase in RPE cells, and it catalyzes the rate-limiting step of the visual cycle converting all-trans-retinyl esters to 11-cis-retinol. PBN efficiently inhibited this activity with an IC₅₀ of 0.1 mM (FIG. 7) and significantly decreased the supply of 11-cis-retinal to the photoreceptors in the retina. Thus, by inhibiting RPE65, PBN reduces the rate of visual pigment regeneration and substantially decreases the quantity of bleachable rhodopsin (FIG. 5). Therefore, it appears that since PBN reduces the quantity of the primary and necessary trigger for light-damage (i.e., bleachable rhodopsin), this is the mechanism by which it provides significant protection from light damage. This also explains why PBN is not effective when administered after light exposure (FIGS. 1 and 2), because light-damaging rhodopsin activation was already induced. The effect of PBN on rhodopsin regeneration was observed to last for several hours (FIG. 5) and could prevent light-damage when administered up to 12 hours before the exposure. An additional potentially beneficial effect of PBN on scavenging the oxidants generated by intense light exposure cannot be ruled out; however, given the profound effect on RPE65 activity and on rhodopsin regeneration, this appears to be the primary protective mechanism in light damage.

Although PBN clearly and potently inhibited rhodopsin regeneration and recovery of rod photoreceptor responsiveness after bleaching, cone function was maintained in the presence of PBN. These results support a growing body of evidence indicating a non-canonical visual cycle in the retina that rapidly provides 11-cis-retinaldehyde to cones (reviewed recently in (58)). Although RPE65 is expressed in mammalian cones (59), it is unclear whether it acts as the isomerohydrolase for the cone-specific visual cycle. Assuming that RPE65 expressed in cones can be inhibited by PBN, the results of the present Example indicate that either RPE65 is not necessary for the retina-specific cone visual cycle or that PBN is preferentially taken up by the RPE and does not reach a high enough concentration in the retina to inhibit the RPE65 expressed in cones. The present Example demonstrates substantial retention of visual function (i.e., normal cone function) under conditions where PBN completely inhibited the canonical, RPE visual cycle. These findings support the use of PBN as a therapeutic agent to slow the accumulation of toxic retinoid intermediates that accumulate in the RPE in diseases such as Stargardt's maculopathies without affecting diurnal vision provided by cone photoreceptors.

The mechanisms of RPE65 inhibition by PBN as well as the mechanism of RPE65 reaction are currently unclear. Recently, it has been proposed that the isomerohydrolase reaction may proceed through the formation of an intermediate retinoid radical cation, which facilitates the retinoid polyene chain isomerization by lowering the energy of activation barrier (60). It is likely that PBN can trap this intermediate radical and interrupt the reaction. Although the spin trapping of retinoid radicals has not been reported in literature, it has been shown that PBN can produce stable adducts with carotenoid radicals (61). Alternatively, PBN can bind at the active site of RPE65, competing with the binding of retinyl ester substrate. To distinguish between these two alternatives, PBN inhibition was studied using chicken recombinant RPE65 in the liposome-based isomerohydrolase assay. PBN inhibits RPE65 uncompetitively (FIG. 8), indicating that it does not bind to the free RPE65 enzyme, but rather binds to the enzyme-substrate complex (44). This supports the idea that PBN may act as a spin trap at the active site of RPE65, binding the retinoid radical intermediate that forms a complex with RPE65.

Retinoid radicals are unstable and most likely can exist only as intermediates at the active site of RPE65. Therefore, to be efficient, the spin trap should have easy access to the active site of RPE65. The RPE65 active site with catalytic iron is located deep inside the hydrophobic tunnel that serves to bind the highly hydrophobic retinyl ester substrate of RPE65 (62). Because of its relatively high hydrophobicity, PBN can penetrate into the active site and interact with the retinoid radical, interrupting the formation of the 11-cis-retinol product. S-PBN is significantly more hydrophilic and probably cannot reach the RPE65 active site due to unfavorable interaction with hydrophobic aromatic residues at the substrate binding tunnel. This may explain why S-PBN does not interfere with RPE65 isomerohydrolase activity and consequently does not protect the retina from light stress. Similar results obtained from an independent study showed that aromatic and more lipophilic nitrone spin traps inhibit RPE65 activity effectively (45).

PBN is a commonly used free radical spin trap. Several derivatives of PBN are in pre-clinical trials for protection of tissues from oxidative damage, such as observed in stroke and ischemic injuries. The present Example indicates that PBN might be a good therapeutic candidate for human retinal degenerations. Interference with the normal visual cycle is one of the strategies for treating Stargardt's diseases. This approach could preserve vision by decreasing the accumulation of toxic retinoid metabolites, such as the retinal fluorophore A2E, a major component of lipofuscin and a side product of the visual cycle (42). Following a similar strategy, there are at least two other compounds entered in human clinical trials, fenretinide-N-(4-hydroxyphenyl)retinamide (Sirion Therapeutics, Tampa, Fla.) and ACU-4429 (Acucela, Bothell, Wash.) demonstrating the potential of this therapeutic strategy (63-64). The present Example illustrates that PBN did not inhibit cone visual cycle and thus is not anticipated to affect diurnal vision if taken as a drug. However, it is noted that its effect on rod visual function may result in reduced night vision, as has been observed for other inhibitors of the visual cycle (53, 64-65). PBN is already a proven compound for photoreceptor preservation, and this determination of its mechanism of action makes it a potential agent for targeted therapies for Stargardt's disease and dry AMD in which accumulation of A2E leads to pathogenesis.

### Example 3

In this Example, the effect of several PBN analogs on RPE65 isomerohydrolase activity was investigated in an in vitro assay. The chemical structures of the PBN analogs utilized are described herein above. Bovine RPE microsomes were used as a source of the RPE65 protein and all-trans-[³H] retinol was a substrate for the isomerohydrolase assay. The produced 11-cis-[³H] retinol was quantified by normal phase HPLC with flow scintillation analyzer. The inhibition of isomerohydrolase activity was dependent on the PBN analog's concentration.

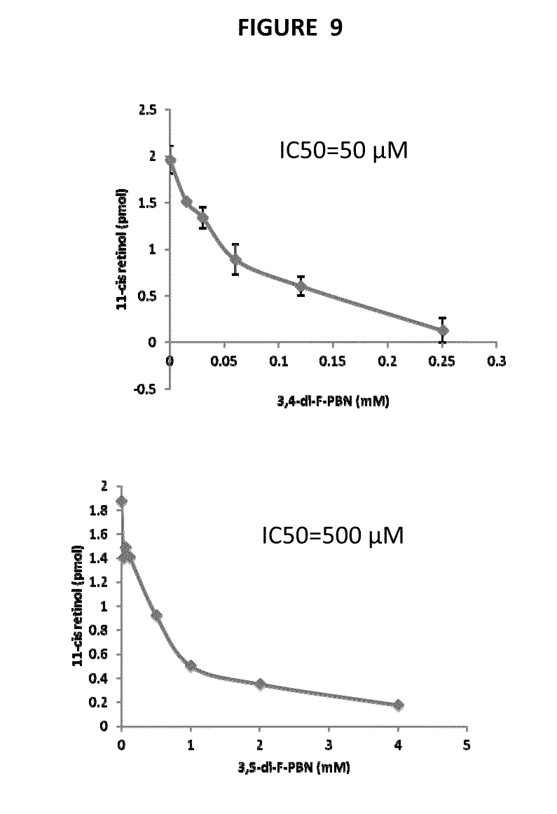

The calculated apparent IC₅₀ values for the assayed PBN analogs are shown in Table 1. FIG. 9 also illustrates the calculation of IC₅₀ values for the two difluoro-PBN analogs. IC₅₀ values are the concentration of the inhibitor that inhibits 50% of the enzymatic activity. Therefore, the smaller the IC₅₀ value, the better the drug.

As can be seen from Table 1, the fluorinated analogs 4-5-PBN, 4-CF₃-PBN, and 3,4-di-F-PBN were the most efficient inhibitors. In fact, these three analogs were more effective at inhibiting RPE65 activity than the native PBN molecule.

TABLE 1
IC₅₀ Values for the Inhibition of
RPE65 Isomerohydrolase Reaction by the PBN Analogs

| Compound | Compound ID | IC₅₀ (μM) |
|---|---|---|
| PBN | 1 | 100 |
| 4-F-PBN | 2 | 60 |
| 4-CF₃-PBN | 3 | 60 |
| 4-Me-PBN | 4 | 100 |
| 4-OMe-PBN | 5 | 500 |
| 4-OEt-PBN | 6 | >2000 |
| 2-F-PBN | 7 | 1300 |
| 3-F-PBN | 8 | 280 |
| 3,5-di-F-PBN | 9 | 500 |
| 3,4-di-F-PBN | 10 | 50 |
| S-PBN | 11 | >3000 |

### Example 4

In this Example, the effects of PBN derivatives on retinal protection from light-induced damage were tested in vivo. Three of the PBN derivatives from Example 3—4-fluoro-PBN (4-F-PBN), 4-trifluoromethyl-PBN (4-CF₃-PBN), and 4-methyl-PBN (4-Me-PBN)—were administered to rats, and their effects on rhodopsin regeneration and protection from light damage were evaluated via ERG and histological analysis.

The PBN derivatives were injected intraperitoneally (IP) into albino SD rats at 50 mg/kg body weight at 0.5 hours before the start of light-damage exposure at 2,700 lux for 6 hours. After the exposure, the rats were moved to their dim cyclic room to recover for a week. Retinal damage/protection was assessed by functional analysis by ERG and structural analysis of histological sections. Dark adapted (12 hour) and Light adapted (for 2 hour) retinas were utilized as controls.

A. Inhibition of Regeneration of Rhodopsin

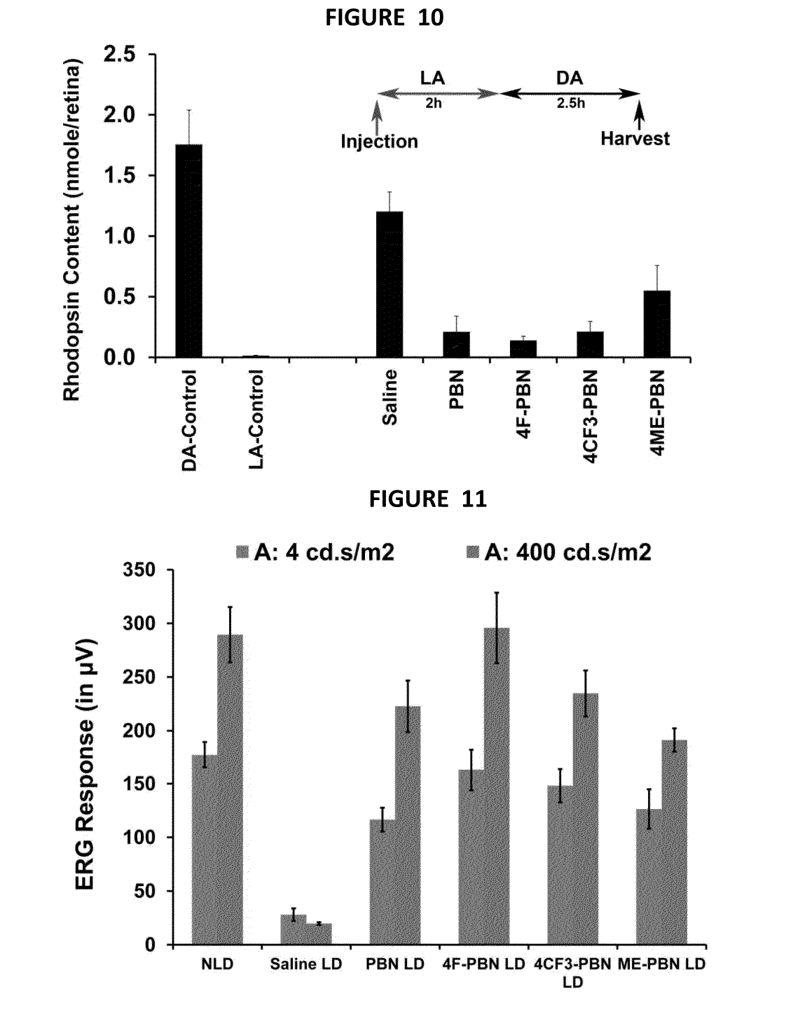

FIG. 10 illustrates the effects of the PBN derivatives on rhodopsin regeneration. Saline treated and dark adapted control retinas recovered approximately 75% rhodopsin following 2.5 hours in the dark. However, PBN and all three derivatives blocked rhodopsin regeneration significantly.

The % inhibition of regeneration of rhodopsin data for the representative compounds tested are given in Table 2. As can be seen from Table 2, PBN and the analogs 4-F-PBN, 4-CF₃-PBN, and 4-Me-PBN were the most efficient inhibitors of rhodopsin regeneration.

TABLE 2
Inhibition of Regeneration of Rhodopsin

| Compound | Compound ID | % Inhibition |
|---|---|---|
| | Vehicle | 0 |
| PBN | 1 | 83 |
| 4-F-PBN | 2 | 88 |
| 4-CF₃-PBN | 3 | 82 |
| 4-Me-PBN | 4 | 54 |

B. Protection of Retinal Function

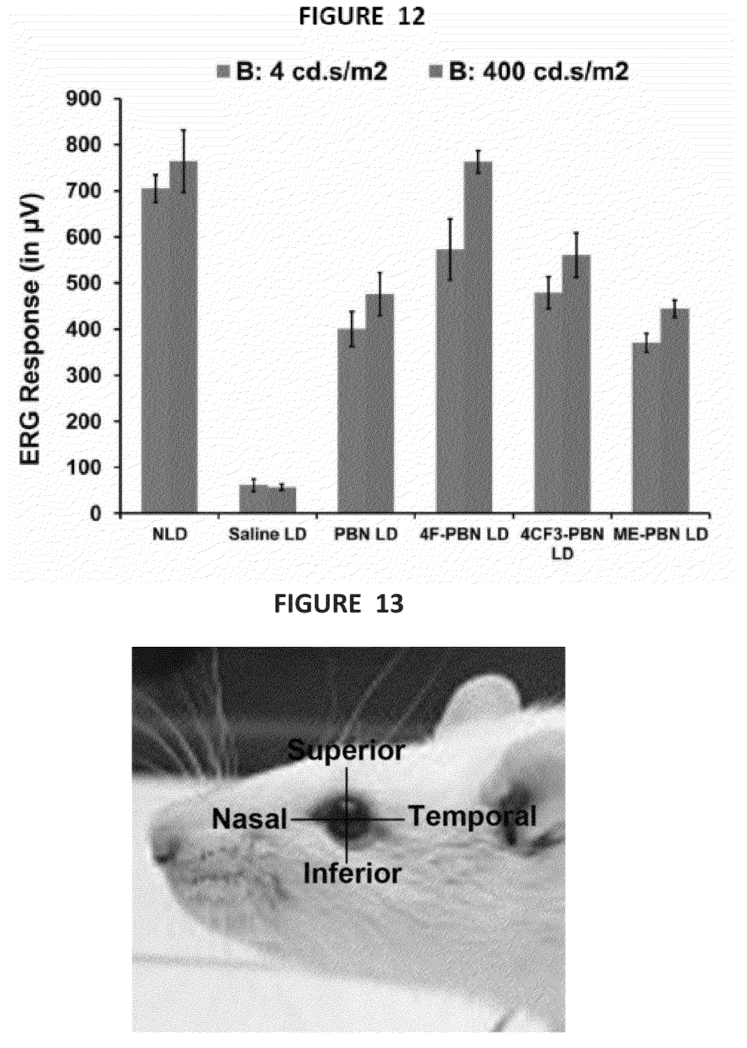

FIGS. 11-12 illustrate ERG A-wave and B-wave analyses. Electroretinographic (ERG) responses were recorded for two flash stimuli at intensities of 4 and 400 cd·sec/m². A-wave represents the responses obtained directly from photoreceptor cells, whereas B-wave represents the amplification of A-wave response obtained from secondary neurons in the retina. The dim flash (4 cd·sec/m²) stimulated the rod photoreceptor cells, and the bright flash (400 cd·sec/m²) stimulated both rod and cone photoreceptors. Therefore the blue bars represent only rod responses, and the red bars represent mixed responses from both rod and cone photoreceptors. As seen in FIGS. 11-12, saline treated rats (Saline LD) lost their function significantly, whereas PBN (PBN LD) and PBN-derivatives (4-F-PBN LD, 4-CF₃-PBN LD, 4-Me-PBN LD) protected retinal function significantly.

The electroretinographic (ERG) response data for the representative compounds tested are given in Tables 3 and 4. As can be seen from Table 3, PBN and the analogs 4-F-PBN, 4-CF₃-PBN, and 4-Me-PBN provided the most protection of the retina from light damage.

TABLE 3
Functional Protection of Rat Retina from Light-Damage by
Systemic Administration of Various Nitrones*

| | Com- | A-Wave | % | B-Wave | % |
|---|---|---|---|---|---|
| | pound | value at | Protec- | value at | Protec- |
| Compound | ID | 4 cd · sec/m² | tion | 4 cd · sec/m² | tion |
| | NLD | 177.3 | 100.0 | 704.4 | 100.0 |
| | Saline LD | 28.0 | 0.0 | 60.5 | 0.0 |
| PBN | 1 | 116.5 | 59.3 | 400.4 | 52.8 |
| 4-F-PBN | 2 | 163.0 | 90.4 | 572.4 | 79.5 |
| 4-CF₃-PBN | 3 | 148.1 | 80.4 | 478.7 | 64.9 |
| 4-Me-PBN | 4 | 126.4 | 65.9 | 369.9 | 48.1 |

*measured by ERG at flash intensity 4.0 cd · sec/m² (mostly rod function)

TABLE 4
Functional Protection of Rat Retina From Light-Damage by
Systemic Administration of Various Nitrones**

| | Com- | A-Wave | % | B-Wave | % |
|---|---|---|---|---|---|
| Com- | pound | value at | Protec- | value at | Protec- |
| pound | ID | 400 cd · sec/m² | tion | 400 cd · sec/m² | tion |
| | NLD | 289.4 | 100.0 | 764.2 | 100.0 |
| | Saline | 19.7 | 0.0 | 56.4 | 0.0 |
| | LD | | | | |
| PBN | 1 | 222.7 | 75.3 | 476.1 | 59.3 |
| 4-F-PBN | 2 | 295.7 | 102.3 | 762.6 | 99.8 |
| 4-CF₃- | 3 | 234.7 | 79.7 | 560.0 | 71.2 |
| PBN | | | | | |
| 4-Me- | 4 | 191.3 | 63.6 | 443.9 | 54.7 |
| PBN | | | | | |

**measured by ERG at flash intensity 400.0 cd · sec/m² (mostly rod and cone function)

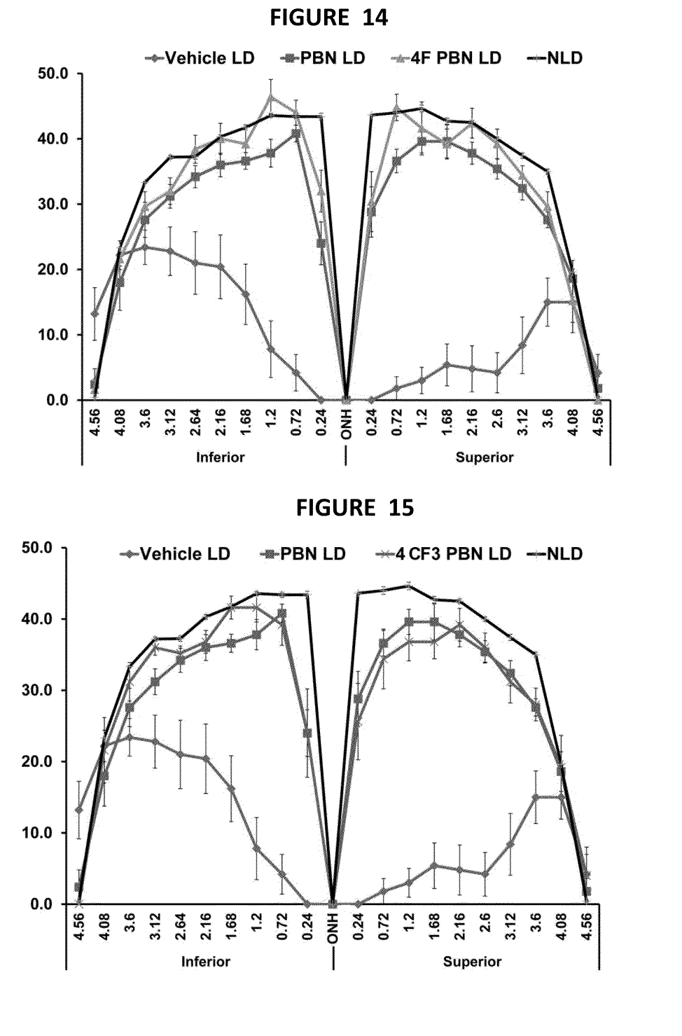

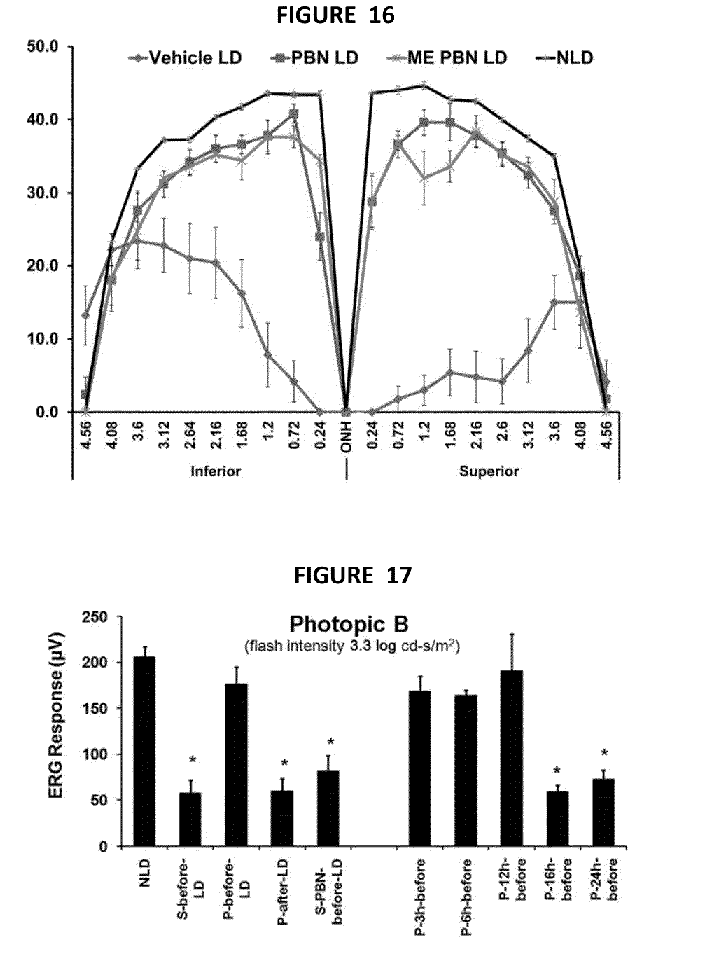

FIGS. 14-16 illustrate the protective effect of the PBN derivatives of retinal photoreceptors. Vehicle or saline treated rats lost most of their photoreceptors from the central retina, and the effect is more pronounced in the superior retina. However, PBN treatment (PBN LD) resulted in retention of approximately 90% photoreceptor cells. In FIG. 14, the effect of 4-F-PBN is comparable to PBN, and even appears to provide more protection than PBN. FIG. 15 illustrates that 4-CF₃-PBN also resulted in a protective effect comparable to that of PBN. FIG. 16 illustrates that 4-Me-PBN also provided significant retinal protection; however, its effect is slightly lower than that observed for PBN.

The data for the structural protection of rat retina from light-damage by systemic administration of various nitrones, estimated by measuring the retinal outer nuclear layer (ONL) thickness in both superior and inferior retina is given in Table 5. The data shows that the superior retina was affected more than the inferior retina in light-damage.

TABLE 5
Structural Protection of Rat Retina from Light-Damage
by Systemic Administration of Various Nitrones

| | | ONL | | ONL | |
|---|---|---|---|---|---|
| | | Thickness | | Thickness | |
| | | (μm) | % | (μm) | % |
| | | Superior | Protec- | Inferior | Protec- |
| Compound | Compound ID | retina | tion | retina | tion |
| | NLD | 43.5 ± 0.5 | 100.0 | 42.3 ± 0.8 | 100.0 |
| | Saline LD | 3.8 ± 1.3 | 0.0 | 12.2 ± 2.3 | 0.0 |
| PBN | 1 | 38.4 ± 1.0 | 87.1 | 37.8 ± 0.9 | 85.0 |
| 4-F-PBN | 2 | 42.0 ± 1.4 | 96.2 | 42.4 ± 1.3 | 100 |
| 4-CF₃-PBN | 3 | 36.8 ± 1.4 | 83.1 | 39.8 ± 1.1 | 91.6 |
| 4-Me-PBN | 4 | 35.2 ± 1.2 | 79.1 | 36.2 ± 1.0 | 79.7 |

### Example 5

U.S. Pat. No. 5,622,994, issued Apr. 22, 1997 to Carney and Floyd; U.S. Pat. No. 6,002,001, issued Dec. 14, 1999 to Carney and Floyd; and US Patent Application Publication No. US 2010/0168112 A1, published Jul. 1, 2010 to Kelly et al. disclose compositions that are used in accordance with presently disclosed and claimed inventive concept(s). These patents/publications are expressly incorporated herein by reference in their entireties attached hereto, and thus it is to be understood that the entire contents of each of the referenced patents or publications is included within this Specification.

The nitrone compounds of the above references are examined in vitro by the methods described in Example 3 and in vivo by the methods described in Example 4. The effects of the compounds on retinal protection from light-induced damage are determined in vivo, and compounds that are comparable in activity to PBN (or possess greater activity than PBN) are identified.

### Example 6

This Example uses nitrone compounds of the Formula I:

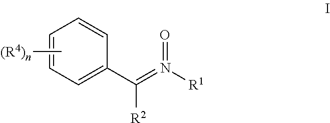

wherein R¹ is substituted or unsubstituted alkyl; R² is H, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl; R⁴ is selected from halo, alkyl, haloalkyl, substituted or unsubstituted alkoxy, alkoxyalkyl, cyano, nitro, SO₃H, SOR^{4a}, SO₂R^{4a}, SO₂NR^{4a}R^{4b}; R^{4a} and R^{4b} is H, substituted or unsubstituted alkyl, substituted or unsubstituted aryl, or substituted or unsubstituted heteroaryl; or R^{4a} and R^{4b} taken together with the N they are attached to form heterocycle; and n is 1, 2, 3, or 4; provided that when n is 1, and R^{4a} is 2-SO₃H; then R¹ is other than t-Bu.

Particular nitrone compounds used in this method include: (a) wherein R² is H; (b) wherein R⁴ is halo, cyano, methoxy, CF₃, OCF₃, substituted or unsubstituted alkyl, C₁-C₆ alkyl, Me, Et, n-Pr, i-Pr, n-Bu, t-Bu, C₂-C₆ alkoxy, —OMe, -OEt, —O-n-Pr, —O-i-Pr, —O-n-Bu, or —O-t-Bu; R² is H; and R¹ is other than t-Bu; (c) wherein n is 2; and each R⁴ is SO₃H; and (d) wherein R⁴ is SOR^{4a}, or SO₂R^{4a}, or SO₂NR^{4a}R^{4b}; and wherein R^{4b} is H, or alkyl, Me, Et, n-Pr, i-Pr, n-Bu, or t-Bu; and wherein R^{4a} and R^{4b} taken together with the N they are attached to form heterocycle, pyrrolidinyl, piperidinyl, morpholinyl, or piperazinyl; and wherein R^{4a} is H, alkyl, aryl, or heteroaryl, Me, Et, n-Pr, i-Pr, n-Bu, or t-Bu, Ph, or pyridyl.

The nitrone compounds of this Example are examined in vitro by the methods described in Example 3 and in vivo by the methods described in Example 4. The effects of the compounds on retinal protection from light-induced damage are determined in vivo, and compounds that are comparable in activity to PBN (or possess greater activity than PBN) are identified. Any of the compounds of the present example may be used in any of the methods described elsewhere herein.

### Example 7

### Topically Administered PBN Reaches the RPE and Inhibits Rhodopsin Regeneration

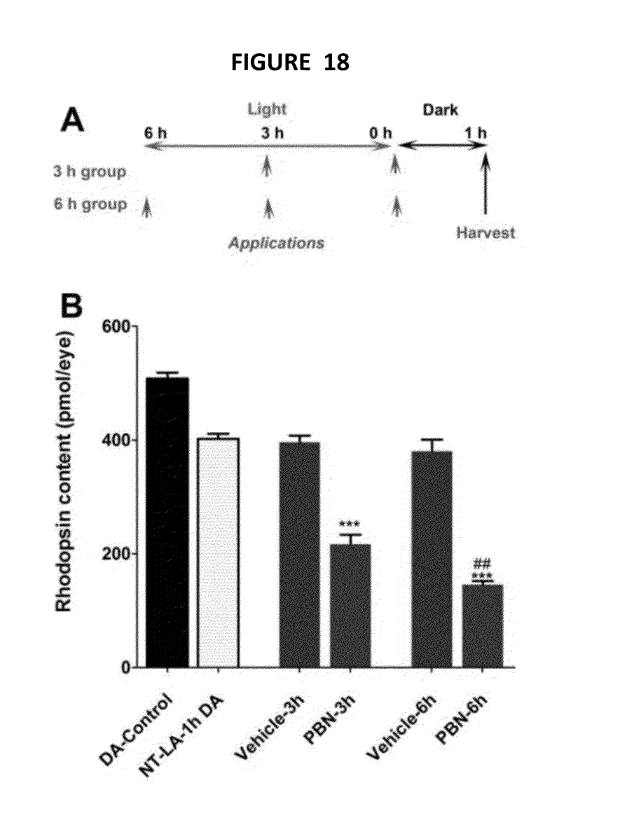

An eye drop formulation was used with various absorption enhancers for topical application of PBN to mouse eyes. When one drop (˜50 μl) containing 10% PBN was applied to completely light-adapted mouse eyes and the mouse moved to the dark for 1 h, the regeneration of bleachable rhodopsin was significantly inhibited (FIGS. 18A and 18B). One hour dark adaptation followed by light causes 75-80% recovery of bleachable rhodopsin in the Balb/c mice (FIG. 18B, NT-LA 1 h DA). The eye drop without PBN (vehicle) had no effect on rhodopsin regeneration (FIG. 18B, vehicle). However, both 3 and 2 applications at 6, 3, and 0 h and at 3 and 0 h before dark adaptation, respectively, inhibited rhodopsin regeneration significantly, and inhibition from 3 applications was greater than 2 applications (FIGS. 18A and 18B). This result indicates: 1) Topically applied PBN reaches the RPE and slows down the visual cycle, 2) PBN action could be dose dependent, 3) PBN action could be time dependent, and 4) There may be a time lag for PBN to reach and accumulate in the RPE cells to reach a threshold level to inhibit RPE65.PBN is detected inside the eye by mass spectrometry.

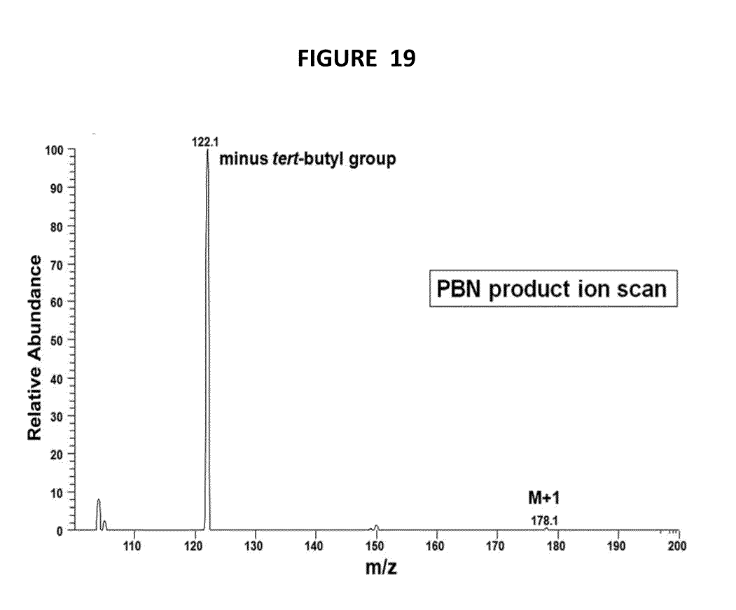

The presence of PBN in the posterior eye cup was conclusively demonstrated by mass spectrometry (FIG. 19). Eyes from mice given one drop of PBN solution or vehicle at 6, 3, and 0 h before euthanasia were removed, washed extensively, and the posterior eye cup extracted and PBN presence determined. The MS tracing shown in FIG. 19 is of PBN found in the eye cup following 3 separate doses. No PBN was found in the eye cups of the vehicle treated control group. The MS result confirms that PBN applied topically reaches the RPE.

Topically administered PBN protects the retina from light damage

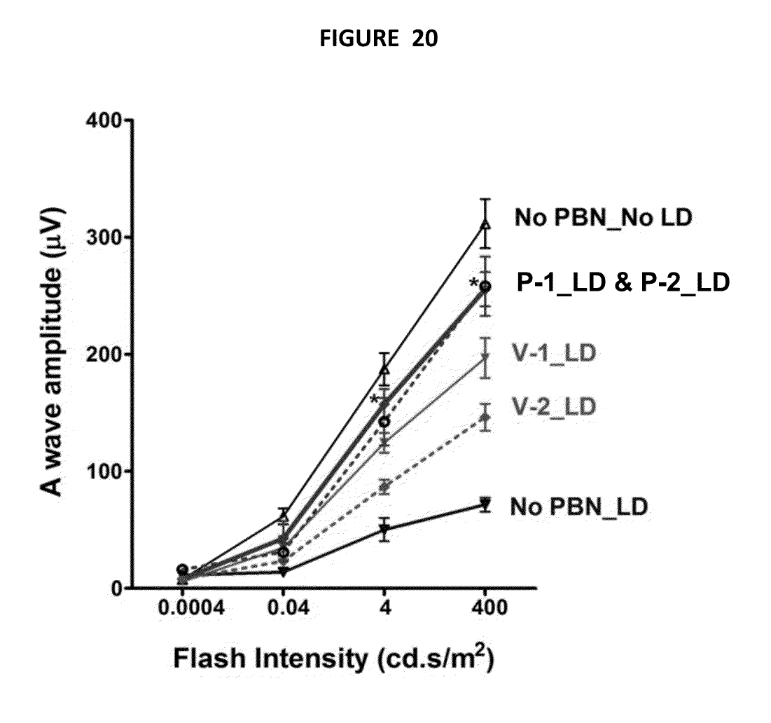

FIG. 20 shows that topically-applied PBN protects mouse retina from light-damage (LD). After topical application of PBN, light-damage was done at 3,000 lux for 6 h. ERG responses were recorded five days after light-damage. Scotopic A-wave amplitudes were measured at increasing flash intensity and presented in the figure to show rod function. Two vehicles were used to solubilize and make the 10% PBN eye drop formulation for topical application on mouse eyes, vehicle 1 (V-1) and vehicle 2 (V-2). Upon light-damage, A-wave amplitudes decreased significantly in untreated control eyes (No PBN_LD), however, significant restoration of rod function was observed in PBN treated eyes, in which PBN was solubilized either in V1 or V2 (P-1_LD and P-2_LD). Vehicles showed slight protection (V-1_LD and V-2_LD) but PBN protection was higher than that provided by either vehicle (* p<0.05, n=6). Vehicle 2 did not induce as much of a protective response by itself as that seen when Vehicle 1 was used. Vehicle 1 contains 10 mM potassium borate/bicarbonate buffer (pH 7.2), glycerine, 1.0%, carboxymethylcellulose 0.3%, benzyl alcohol 0.3%, and 30% 1-methyl-2-pyrrolidinone (a solubilizing enhancer, a.k.a., N-Methyl-2-Pyrrolidone—“NMP”). Vehicle 2 contains 137 mM sodium chloride, 2.7 mM potassium chloride in 10 mM phosphate buffer (pH 7.4), and 30% 1-methyl-2-pyrrolidinone.

### Example 8

### PBN Delivery and Physiological Effects after Topical Application in Baboons

PBN (10 mg/ml) in vehicle 2 was applied to eyes of two anesthetized baboons. Dosage was two drops every 15 minutes for 1¾ hours (total of 8 doses). Vehicle was applied to the right eyes using the same schedule. Two hours after the first drops, the eyes were thoroughly flushed with vehicle and enucleated, and rinsed again with vehicle. Eyes were wrapped in aluminum foil and placed on ice for 2 hours, after which each eye was hemisected under dim red light and the anterior segment discarded. The retina was dissected from the retinal pigment epithelium (RPE) and suspended in a petri dish containing PBS. Three-four pieces of tissue from each retina were removed (still under dim red light) and placed into individual plastic tubes, which were wrapped in aluminum foil and frozen immediately. These were used to determine rhodopsin regeneration (Table 6). Retinas and posterior eyecups were brought into room light. Four small cuts were made in the eyecups so that they could lie flat in a petri dish. Small pieces of RPE/choroid were dissected from each quadrant for MS/MS analysis of PBN.

Under dim red light, pieces of dark-adapted retinas were homogenized in 450 μl of buffer containing 10 mM Tris-HCl (pH 7.4), 150 mM NaCl, 1 mM EDTA, 2% (w/v) octylglucoside, and 50 mM hydroxylamine. Homogenates were centrifuged at 16,000 g and soluble lysates were scanned from 270-800 nm in a spectrophotometer (Ultrospec 3000 UV/Vis Spectrophotometer, GE Healthcare, Piscataway, N.J.). Samples were then bleached under room light for 10 min and scanned again. The difference spectra at 500 nm between pre- and post-bleached samples were used to determine rhodopsin content using a molar extinction coefficient of 42,000 M⁻¹. Protein values were determined by absorbance at 280 nm and the rhodopsin content calculated as picomoles/mg protein. The percent regeneration in PBN-treated eyes was determined relative to values for the vehicle treated eyes, which were set at 100%.

Pieces of RPE/choroid were weighed and homogenized in 40% methanol_{(aq)}. The PBN derivative 4-F-PBN was added as an internal standard (0.1 μg). The homogenates were diluted with chloroform:methanol:isopropanol 1:2:4 containing 20 mM ammonium formate. Samples were vortexed, centrifuged, and aliquoted into Eppendorf twin-tec 96-well plates (Thermo Fisher Scientific, Inc., San Jose, Calif.) and sealed with Corning Thermowell sealing tape (Thermo Fisher Scientific, Inc.). Samples were introduced into a triple quadrupole mass spectrometer (TSQ Ultra, Thermo Fisher Scientific Inc.) through a chip-based nano-ESI source (NanoMate®, Advion, Inc., Ithaca, N.Y.) operating in infusion mode. Chipsoft version 8.3.3 software (Advion, Inc.) was used to set the NanoMate spray voltage to 1.4 kV and the gas pressure to 0.6 psi. The ion transfer tube of the mass spectrometer was maintained at 192° C. MS/MS spectra were acquired at a rate of 500 m/z per second by methods created using XCalibur software (Thermo Fisher Scientific, Inc.). Analytes were measured in SRM (single reaction monitoring) mode with m/z 178 to 122 for PBN and m/z 196 to 140 for 4-F-PBN. The isolation windows of quadrupoles 1 and 3 were maintained at 0.5 Da, while the collision gas pressure of quadrupole 2 was maintained at 1.0 mtorr of argon. Collision energy was optimized at 20 V. Concentrations were calculated using XCalibur software (Thermo Fisher Scientific, Inc.).

TABLE 6
Rhodopsin Regeneration Inhibition by PBN in Baboons

| Baboon | Percent rhodopsin | |
|---|---|---|
| number and | regeneration | PBN level in RPE/choroid |
| treatment | relative to control | (ng/mg wet weight) |
| 1-Vehicle | 100 (Avg of 2 samples | 0 (Avg of 3 samples |
| | from one eye) | from one eye) |
| 1-PBN | 26 (Avg of 3 samples | 24.2 (Avg of 3 samples |
| | from one eye) | from one eye) |
| 2-Vehicle | 100 (Avg of 6 samples | 0 (Avg of 4 samples |
| | from one eye) | from one eye) |
| 2-PBN | 36 (Avg of 6 samples | 23.5 (Avg of 4 samples |
| | from one eye) | from one eye) |

PBN determinations in two baboon eyes clearly show that the compound reached the RPE/choroid. Rhodopsin regeneration was lower in the two eyes treated with PBN compared to controls (Table 6). These results support our finding in mouse eyes treated with PBN, namely that the drug reaches the RPE and inhibits the enzymatic activity of RPE65.

PBN and other nitrones are hydrophobic molecules with limited solubility in water, including intra- and extra-cellular fluids. Their hydrophobic nature increases the difficulty of penetrating the globe of the eye and reaching the retina and RPE. Prior to the present work, the likelihood was low that these molecules when applied topically could reach the target cells inside of the eye. While amphipathic versions of nitrones with ionizable groups such as phosphonates and sulfonates have increased aqueous solubility, none of these types of nitrones had been previously known to inhibit RPE65 enzymatic activity. Therefore, it was unlikely that PBN or other nitrones shown to inhibit RPE65 activity would be able to penetrate the globe and reach the RPE in sufficient concentration to affect RPE65 enzymatic function. Moreover, organic solvents such as ethanol and dimethyl sulfoxide (DMSO) cannot be used to solubilize PBN and other nitrones because they would injure the cornea, sclera, and conjunctiva. However, the present work went forward in spite of the unlikelihood of success. In the present work two delivery vehicles (V-1 and V-2) were used to solubilize PBN for intraocular delivery. Both vehicles are shown herein to facilitate PBN delivery to the RPE in sufficient quantities to affect RPE65 activity. Vehicle 1 was used in the mouse study and slowed rhodopsin regeneration and recovery of the ERG response, and protected from light stress-induced retinal degeneration. Vehicle 2 facilitated PBN delivery to the RPE of baboon eyes and slowed the regeneration of rhodopsin. Delivery of PBN to target tissues by both vehicles was confirmed by mass spectrometric analysis of the tissues. Other solubilizing enhancers that can be used in place of NMP include, but are not limited to, 2-pyrrolidone, dimethylformamide, and dimethylacetamide.

In one embodiment, the inventive concept(s) is a method of treating an eye disease in a subject, comprising: obtaining a composition comprising (1) an inhibitor of retinal pigment epithelium-specific protein 65 kDa (RPE65), and (2) an aqueous delivery vehicle able to solubilize the RPE65 inhibitor; and topically applying the composition to the eye of a subject in need of such treatment. The RPE65 inhibitor may be, for example, alpha-phenyl-N-tert-butyl nitrone or a salt, ester, derivative, or analog thereof, as described elsewhere herein. In the method, rhodopsin regeneration in the retinal pigment epithelium is reduced by at least about 50%, 60%, 70%, 80%, 90%, or more.

### Example 9

Seven PBN derivatives were provided in eye drop formulations and tested in vivo via topical application for their effectiveness in inhibiting rhodopsin regeneration. Rhodopsin recovery indirectly measures the inhibition of RPE65 enzyme activity. Percent rhodopsin recovery can therefore be compared with the in vitro IC₅₀ values to determine the inhibition of RPE65 isomerohydrolase reaction.

Eye drop formulations were prepared as described in Example 7 and contained 5% or 10% of a PBN derivative. The eye drop formulations were applied to Balb/c mice by the 6 h group application scheme shown in FIG. 18A, in which the eye drops were applied at 6, 3, and 0 h before dark adaptation. The eye drops were applied to completely light-adapted mouse eyes, and the mice were then moved to the dark for 1 h to allow the regeneration of bleachable rhodopsin. The results are shown in Table 7. One hour dark adaptation followed by light causes 75-82% recovery of bleachable rhodopsin in untreated mice (00, No Treatment). The eye drop without any nitrones (vehicle) had no effect on rhodopsin regeneration (000, vehicle). PBN inhibited rhodopsin regeneration significantly. In addition, three other nitrones, 4-CF₃-PBN, 4-Me-PBN and 3,4-di-F-PBN, are more effective in inhibiting rhodopsin regeneration than PBN when administered in this eye drop formulation.

As shown in Table 7, most of the compounds with IC₅₀ values similar to or lower than PBN exhibited a similar effect to PBN in inhibiting rhodopsin regeneration when administered as an eye drop. However, 4-F-PBN, which demonstrated an IC₅₀ below PBN, was not effective at inhibiting rhodopsin regeneration when administered in this particular eyedrop formulation; this data suggests that a different eye drop formulation may be required for 4-F-PBN to be effective as an eye drop.

TABLE 7
Inhibition of Rhodopsin Regeneration Compared to IC₅₀ Values
For Various Nitrone Eye Drop Formulations

| Com- | | | | % in | % of |
|---|---|---|---|---|---|
| pound | | Expt. | IC₅₀ | Eye | Rhodopsin |
| ID | Compound | Condition | (μM) | drop | recovery |
| 0 | No | DA | — | — | 100 |
| | Treatment | | | | |
| 00 | No | LA + 1 h DA | — | — | 81.34 |
| | Treatment | | | | |
| 000 | Vehicle | LA + 1 h DA | — | 100 | 78.83 |
| 1 | PBN | LA + 1 h DA | 100 | 10 | 48.67 |
| 2 | 4-F-PBN | LA + 1 h DA | 60 | 5 | 81.15 |
| 3 | 4-CF₃-PBN | LA + 1 h DA | 60 | 5 | 52.80 |
| 4 | 4-Me-PBN | LA + 1 h DA | 100 | 5 | 32.54 |
| 5 | 4-OMe-PBN | LA + 1 h DA | 500 | 5 | 79.10 |
| 6 | 4-OEt-PBN | LA + 1 h DA | >2000 | 5 | 81.99 |
| 7 | 2-F-PBN | | 1300 | | NT |
| 8 | 3-F-PBN | | 280 | | NT |
| 9 | 3,5-di-F-PBN | | 500 | | NT |
| 10 | 3,4-di-F-PBN | LA + 1 h DA | 50 | 5 | 39.31 |
| 11 | S_PBN | LA + 1 h DA | >3000 | 5 | 84.64 |

NT, not tested

Thus, in accordance with the presently disclosed and claimed inventive concept(s), there has been provided a method of inhibiting retinal degeneration, as well as compositions useful in said method, that fully satisfy the objectives and advantages set forth hereinabove. Although the presently disclosed and claimed inventive concept(s) has been described in conjunction with the specific drawings, experimentation, results and language set forth hereinabove, it is evident that many alternatives, modifications, and variations will be apparent to those of ordinary skill in the art. Accordingly, it is intended to embrace all such alternatives, modifications and variations that fall within the spirit and broad scope of the presently disclosed and claimed inventive concept(s).

### REFERENCES

The following references, to the extent that they provide exemplary procedural or other details supplementary to those set forth herein, are specifically incorporated herein by reference.
1. Ranchon, I., Chen, S., Alvarez, K., and Anderson, R. E. (2001) Invest. Ophthalmol. Vis. Sci. 42, 1375-1379
2. Ranchon, I., LaVail, M. M., Kotake, Y., and Anderson, R. E. (2003) J. Neurosci. 23, 6050-6057
3. Tanito, M., Elliott, M. H., Kotake, Y., and Anderson, R. E. (2005) Invest. Ophthalmol. Vis. Sci. 46, 3859-3868
4. Tanito, M., Kaidzu, S., and Anderson, R. E. (2007) Invest. Ophthalmol. Vis. Sci. 48, 1864-1872
5. Tomita, H., Kotake, Y., and Anderson, R. E. (2005) Invest. Ophthalmol. Vis. Sci. 46, 427-434
6. Miyajima, T., and Kotake, Y. (1995) Biochem. Biophys. Res. Commun. 215, 114-121
7. Novelli, G. P., Angiolini, P., Tani, R., Consales, G., and Bordi, L. (1986) Free. Radic. Res. Commun. 1, 321-327
8. Hamburger, S. A., and McCay, P. B. (1989) Circ. Shock 29, 329-334
9. Carney, J. M., Starke-Reed, P. E., Oliver, C. N., Landum, R. W., Cheng, M. S., Wu, J. F., and Floyd, R. A. (1991) Proc. Natl. Acad. Sci. U.S.A. 88, 3633-3636
10. Phillis, J. W., and Clough-Helfman, C. (1990) Neurosci. Lett. 116, 315-319
11. Tabatabaie, T., Kotake, Y., Wallis, G., Jacob, J. M., and Floyd, R. A. (1997) FEBS letters 407, 148-152
12. Zarbin, M. A. (2004) Arch. Ophthalmol. 122, 598-614
13. Saito, K., Yoshioka, H., and Cutler, R. G. (1998) Biosci. Biotechnol. Biochem. 62, 792-794
14. Sack, C. A., Socci, D. J., Crandall, B. M., and Arendash, G. W. (1996) Neurosci. Lett. 205, 181-184
15. De La Paz, M. A., and Anderson, R. E. (1992) Invest. Ophthalmol. Vis. Sci. 33, 2091-2096
16. Organisciak, D. T., Darrow, R. M., Jiang, Y. I., Marak, G. E., and Blanks, J. C. (1992) Invest. Ophthalmol. Vis. Sci. 33, 1599-1609
17. Organisciak, D. T., Wang, H. M., Li, Z. Y., and Tso, M. O. (1985) Invest. Ophthalmol. Vis. Sci. 26, 1580-1588
18. Wiegand, R. D., Giusto, N. M., Rapp, L. M., and Anderson, R. E. (1983) Invest. Ophthalmol. Vis. Sci. 24, 1433-1435
19. Kotake, Y., Sang, H., Miyajima, T., and Wallis, G. L. (1998) Biochim. Biophys. Acta. 1448, 77-84
20. Sang, H., Wallis, G. L., Stewart, C. A., and Kotake, Y. (1999) Arch. Biochem. Biophys. 363, 341-348
21. Hafezi, F., Steinbach, J. P., Marti, A., Munz, K., Wang, Z. Q., Wagner, E. F., Aguzzi, A., and Reme, C. E. (1997) Nat. Med. 3, 346-349
22. Hao, W., Wenzel, A., Obin, M. S., Chen, C. K., Brill, E., Krasnoperova, N. V., Eversole-Cire, P., Kleyner, Y., Taylor, A., Simon, M. I., Grimm, C., Reme, C. E., and Lem, J. (2002) Nat. Genet. 32, 254-260
23. Organisciak, D. T., and Vaughan, D. K. (2010) Prog. Retin. Eye Res. 29, 113-134
24. Jin, M., Li, S., Moghrabi, W. N., Sun, H., and Travis, G. H. (2005) Cell 122, 449-459
25. Moiseyev, G., Chen, Y., Takahashi, Y., Wu, B. X., and Ma, J. X. (2005) Proc. Natl. Acad. Sci. U.S.A. 102, 12413-12418
26. Redmond, T. M., Poliakov, E., Yu, S., Tsai, J. Y., Lu, Z., and Gentleman, S. (2005) Proc. Natl. Acad. Sci. U.S.A. 102, 13658-13663
27. Gu, S. M., Thompson, D. A., Srikumari, C. R., Lorenz, B., Finckh, U., Nicoletti, A., Murthy, K. R., Rathmann, M., Kumaramanickavel, G., Denton, M. J., and Gal, A. (1997) Nat. Genet. 17, 194-197
28. Marlhens, F., Bareil, C., Griffoin, J. M., Zrenner, E., Amalric, P., Eliaou, C., Liu, S. Y., Harris, E., Redmond, T. M., Arnaud, B., Claustres, M., and Hamel, C. P. (1997) Nat. Genet. 17, 139-141
29. Morimura, H., Fishman, G. A., Grover, S. A., Fulton, A. B., Berson, E. L., and Dryja, T. P. (1998) Proc. Natl. Acad. Sci. U.S.A. 95, 3088-3093
30. Lorenz, B., Gyurus, P., Preising, M., Bremser, D., Gu, S., Andrassi, M., Guth, C., and Gal, A. (2000) Invest. Ophthalmol. Vis. Sci. 41, 2735-2742
31. Thompson, D. A., Gyurus, P., Fleischer, L. L., Bingham, E. L., McHenry, C. L., Apfelstedt-Sylla, E., Zrenner, E., Lorenz, B., Richards, J. E., Jacobson, S. G., Sieving, P. A., and Gal, A. (2000) Invest. Ophthalmol. Vis. Sci. 41, 4293-4299
32. Fan, J., Rohrer, B., Moiseyev, G., Ma, J. X., and Crouch, R. K. (2003) Proc. Natl. Acad. Sci. U.S.A. 100, 13662-13667
33. Redmond, T. M., Yu, S., Lee, E., Bok, D., Hamasaki, D., Chen, N., Goletz, P., Ma, J. X., Crouch, R. K., and Pfeifer, K. (1998) Nat. Genet. 20, 344-351
34. Grimm, C., Wenzel, A., Hafezi, F., Yu, S., Redmond, T. M., and Reme, C. E. (2000) Nat. Genet. 25, 63-66
35. Williams, H. E., Claybourn, M., and Green, A. R. (2007) Free Radic Res 41, 1047-1052
36. Tanito, M., Brush, R. S., Elliott, M. H., Wicker, L. D., Henry, K. R., and Anderson, R. E. (2009) J. Lipid Res. 50, 807-819
37. Irreverre, F., Stone, A. L., Shichi, H., and Lewis, M. S. (1969) J. Biol. Chem. 244, 529-536
38. Moiseyev, G., Crouch, R. K., Goletz, P., Oatis, J., Jr., Redmond, T. M., and Ma, J. X. (2003) Biochemistry 42, 2229-2238
39. Nikolaeva, 0., Takahashi, Y., Moiseyev, G., and Ma, J. X. (2009) FEBS. J. 276, 3020-3030
40. Weng, J., Mata, N. L., Azarian, S. M., Tzekov, R. T., Birch, D. G., and Travis, G. H. (1999) Cell 98, 13-23
41. Kasus-Jacobi, A., Ou, J., Birch, D. G., Locke, K. G., Shelton, J. M., Richardson, J. A., Murphy, A. J., Valenzuela, D. M., Yancopoulos, G. D., and Edwards, A. O. (2005) J. Biol. Chem. 280, 20413-20420
42. Maiti, P., Kong, J., Kim, S. R., Sparrow, J. R., Allikmets, R., and Rando, R. R. (2006) Biochemistry 45, 852-860
43. Moiseyev, G., Takahashi, Y., Chen, Y., Kim, S., and Ma, J. X. (2008) J. Biol. Chem. 283, 8110-8117
44. Fersht, A. (1985) Enzyme structure and Mechanism, W.H. Freeman and Company, New York
45. Poliakov, E., Parikh, T., Ayele, M., Kuo, S., Chander, P., Gentleman, S., and Redmond, T. M. (2011) Biochemistry (In Press).
46. Reme, C. E., Grimm, C., Hafezi, F., Marti, A., and Wenzel, A. (1998) Prog. Retin. Eye Res. 17, 443-464
47. Wenzel, A., Grimm, C., Seeliger, M. W., Jaissle, G., Hafezi, F., Kretschmer, R., Zrenner, E., and Reme, C. E. (2001) Invest. Ophthalmol. Vis. Sci. 42, 1653-1659
48. Sugawara, T., Sieving, P. A., Iuvone, P. M., and Bush, R. A. (1998) Invest. Ophthalmol. Vis. Sci. 39, 2458-2465
49. Noell, W. K., Walker, V. S., Kang, B. S., and Berman, S. (1966) Invest. Ophthalmol. Vis. Sci. 5, 450-473
50. Humphries, M. M., Rancourt, D., Farrar, G. J., Kenna, P., Hazel, M., Bush, R. A., Sieving, P. A., Sheils, D. M., McNally, N., Creighton, P., Erven, A., Boros, A., Gulya, K., Capecchi, M. R., and Humphries, P. (1997) Nat. Genet. 15, 216-219
51. Noell, W. K., and Albrecht, R. (1971) Science (New York, N.Y. 172, 76-79
52. Noell, W. K., Delmelle, M. C., and Albrecht, R. (1971) Science (New York, N.Y. 172, 72-75
53. Sieving, P. A., Chaudhry, P., Kondo, M., Provenzano, M., Wu, D., Carlson, T. J., Bush, R. A., and Thompson, D. A. (2001) Proc. Natl. Acad. Sci. U.S.A. 98, 1835-1840
54. Gollapalli, D. R., and Rando, R. R. (2004) Proc. Natl. Acad. Sci. U.S.A. 101, 10030-10035
55. Maeda, A., Maeda, T., Golczak, M., Imanishi, Y., Leahy, P., Kubota, R., and Palczewski, K. (2006) Mol. Pharmacol. 70, 1220-1229
56. Wenzel, A., Reme, C. E., Williams, T. P., Hafezi, F., and Grimm, C. (2001) J. Neurosci. 21, 53-58
57. Grimm, C., Wenzel, A., Williams, T., Rol, P., Hafezi, F., and Reme, C. (2001) Invest. Ophthalmol. Vis. Sci. 42, 497-505
58. Wang, J. S., and Kefalov, V. J. (2011) Prog. Retin. Eye Res. 30, 115-128
59. Znoiko, S. L., Crouch, R. K., Moiseyev, G., and Ma, J. X. (2002) Invest. Ophthalmol. Vis. Sci. 43, 1604-1609
60. Redmond, T. M., Poliakov, E., Kuo, S., Chander, P., and Gentleman, S. (2010) J. Biol. Chem. 285, 1919-1927
61. Konovalova, T. A., Kispert, L. D., Polyakov, N. E., and Leshina, T. V. (2000) Free. Radic. Biol. Med. 28, 1030-1038
62. Kiser, P. D., Golczak, M., Lodowski, D. T., Chance, M. R., and Palczewski, K. (2009) Proc. Natl. Acad. Sci. U.S.A. 106, 17325-17330
63. Mata, N. L., and Vogel, R. (2010) Curr. Opin. Ophthalmol. 21, 190-196
64. Kubota, R., Boman, N. L., David, R., Mallikaarjun, S., Patil, S., and Birch, D. (2011) Retina (In Press).
65. Radu, R. A., Mata, N. L., Nusinowitz, S., Liu, X., Sieving, P. A., and Travis, G. H. (2003) Proc. Natl. Acad. Sci. U.S.A. 100, 1742-4747
66. Mandal M N, Moiseyev G P, Elliott M H, Kasus-Jacobi A, Li X, Chen H, Zheng L, Nikolaeva O, Floyd R A, Ma J X, Anderson R E. (2011) Alpha-phenyl-N-tert-butylnitrone (PBN) prevents light-induced degeneration of the retina by inhibiting RPE65 protein isomerohydrolase activity. J Biol. Chem. 286, 32491-501.

What is claimed is:

1. A method for inhibiting RPE65 (retinal pigment epithelium-specific protein of 65 kDa) isomerohydrolase activity in a human retinal pigmented epithelial (RPE) cell, the method comprising the step of contacting the RPE cell with an effective amount of a compound selected from the group consisting of α-phenyl-N-tert-butyl nitrone (PBN); 4-fluoro-PBN (4-F-PBN); 4-trifluoromethyl-PBN (4-CF₃-PBN); 4-methyl-PBN (4-Me-PBN); 3,4-difluoro-PBN (3,4-di-F-PBN); pharmaceutically acceptable salts and stereoisomers thereof; and combinations thereof.

2. The method of claim 1, wherein contacting the cell with the effective amount of the compound reduces lipofuscin in the RPE cell.

3. The method of claim 1, wherein contacting the cell with the effective amount of the compound reduces A2E in the RPE cell.

4. A method of treating an ophthalmic disease or disorder in a subject by inhibiting RPE65 isomerohydrolase activity in at least one RPE cell of the subject, the method comprising the step of administering to the subject a pharmaceutical composition comprising an inhibitor of RPE65 protein isomerohydrolase activity and a pharmaceutically acceptable carrier, wherein the inhibitor of RPE65 protein isomerohydrolase activity is selected from the group consisting of α-phenyl-N-tert-butyl nitrone (PBN); 4-fluoro-PBN (4-F-PBN); 4-trifluoromethyl-PBN (4-CF₃-PBN); 4-methyl-PBN (4-Me-PBN); 3,4-difluoro-PBN (3,4-di-F-PBN); pharmaceutically acceptable salts and stereoisomers thereof; and combinations thereof.

5. The method of claim 4, wherein the ophthalmic condition is selected from the group consisting of glaucoma, macular degeneration, age-related macular degeneration (AMD), Stargardt's disease, diabetic retinopathy, retinal detachment, retinal blood vessel occlusion, retinitis pigmentosa, hemorrhagic retinopathy, retinopathy of prematurity, inflammatory retinal diseases, optic neuropathy, proliferative vitreoretinopathy, retinal dystrophy, ischemia-reperfusion related retinal injury, hereditary optic neuropathy, metabolic optic neuropathy, Sorsby's fundus dystrophy, Best disease, a retinal injury, a retinal disorder associated with viral and/or bacterial infection, a retinal disorder related to light overexposure, retinal disorders associated with other degenerative diseases such as Alzheimer's disease, multiple sclerosis, and Parkinson's disease, or associated with AIDS or other diseases of the brain, and combinations thereof.

6. The method of claim 4, wherein administration of the pharmaceutical composition to the subject inhibits rhodopsin regeneration by 40% to 90% with substantially no inhibition of cone function.

7. The method of claim 4, wherein the ophthalmic condition is associated with the deposition of lipofuscin in RPE cells, and wherein administration of the pharmaceutical composition to the subject reduces lipofuscin in at least one RPE cell of the subject.

8. The method of claim 4, wherein the ophthalmic condition is associated with deposition of A2E in RPE cells, and wherein administration of the pharmaceutical composition to the subject reduces A2E in at least one RPE cell of the subject.

9. The method of claim 4, wherein administration of the pharmaceutical composition to the subject protects retinal function of the subject.

10. The method of claim 4, wherein the pharmaceutical composition is administered via oral, parenteral, intravenous, topical, intravitreal, retrobulbar and/or sub-conjunctival routes.

11. The method of claim 10, wherein the pharmaceutical composition is administered topically to at least one eye of the subject.

12. The method of claim 11, wherein the inhibitor of RPE65 protein isomerohydrolase activity is present in the pharmaceutical composition at a concentration in a range of from about 1% to about 15%.

13. The method of claim 11, wherein the pharmaceutically acceptable carrier comprises a phosphate buffer and a solubilizer.

14. The method of claim 13, wherein the solubilizer is selected from the group consisting of 2-pyrrolidone, N-methyl-2-pyrrolidone (NMP), dimethylformamide, dimethylacetamide, and combinations thereof.

15. An ophthalmic composition for topical application to an eye of a subject, the ophthalmic composition comprising:
an inhibitor of RPE65 protein isomerohydrolase activity selected from the group consisting of α-phenyl-N-tert-butyl nitrone (PBN); 4-fluoro-PBN (4-F-PBN); 4-trifluoromethyl-PBN (4-CF₃-PBN); 4-methyl-PBN (4-Me-PBN); 3,4-difluoro-PBN (3,4-di-F-PBN); pharmaceutically acceptable salts and stereoisomers thereof; and combinations thereof; and
a pharmaceutically acceptable carrier comprising a solubilizer.

16. The ophthalmic composition of claim 15, wherein the inhibitor of RPE65 protein isomerohydrolase activity is present in the ophthalmic composition at a concentration in a range of from about 1% to about 15%.

17. The ophthalmic composition of claim 15, wherein the solubilizer of the pharmaceutically acceptable carrier is selected from the group consisting of 2-pyrrolidone, N-methyl-2-pyrrolidone (NMP), dimethylformamide, dimethylacetamide, and combinations thereof.

18. The ophthalmic composition of claim 17, wherein the pharmaceutically acceptable carrier further comprises a phosphate buffer.

19. The ophthalmic composition of claim 18, wherein the pharmaceutically acceptable carrier is selected from the group consisting of:
(a) 10 mM potassium borate/bicarbonate buffer (pH 7.2), glycerine, 1.0%, carboxymethylcellulose 0.3%, benzyl alcohol 0.3%, and 30% 1-methyl-2-pyrrolidinone; and
(b) 137 mM sodium chloride, 2.7 mM potassium chloride in 10 mM phosphate buffer (pH 7.4), and 30% 1-methyl-2-pyrrolidinone.

20. The ophthalmic composition of claim 15, further defined as an eye drop.
